SEQUENCE LISTING (11 sequences)

SEQ ID NO: 1 - DNA, 576 bp, Ovine
atggcgtgcggggcgacactgaagcggcccatggagttcgaggcggcgctgctgagccctggctctccgaagcggcggcgctgcgcccctctgtccggccccactccgggcctcaggcccccggacgccgaaccgccgccgctgcttcagacgcagaccccaccgccgactctgcagcagcccgccccgcccggcagcgagcggcgccttccaactccggagcaaatttttcagaacataaaacaagaatatagtcgttatcagaggtggagacatttagaagttgttcttaatcagagtgaagcttgtacttcggaaagtcagcctcactcctcagcactcacagcacctagttctccaggttcctcctggatgaaaaaggaccagcccacctttaccctccgacaagttggaataatatgtgagcgtctcttaaaagactatgaagataaaattcgggaggaatatgagcaaatcctcaatactaaactagcagaacaatatgaatcttttgtgaaattcacacatgatcagattatgcgacgatatgggacaaggccaacaagctatgtatcc

SEQ ID NO: 2 - protein, 192 aa, Ovine
MACGATLKRPMEFEAALLSPGSPKRRRCAPLSGPTPGLRPPDAEPPPLLQTQTPPPTLQQPAPPGSERRLPTPEQIFQNIKQEYSRYQRWRHLEVVLNQSEACTSESQPHSSALTAPSSPGSSWMKKDQPTFTLRQVGIICERLLKDYEDKIREEYEQILNTKLAEQYESFVKFTHDQIMRRYGTRPTSYVS

SEQ ID NO: 3 - DNA, 576 bp, Bovine
atggcgtgcggggcgacactgaagcggcccatggagttcgaggcggcgctgctgagccctggctctccgaagcgacggcgctgcgcccctctgtccggccccactccgggcctcaggcccccggacgccgaaccgccaccgctgcttcagacgcagatcccaccgccgactctgcagcagcccgccccgcccggcagcgaccggcgccttccaactccggagcaaatttttcagaacataaaacaagaatatagtcgttatcagaggtggagacatttagaagttgttcttaatcagagtgaagcttgtacttcggaaagtcagcctcactcctcaacactcacagcacctagttctccaggttcctcctggatgaaaaaggaccagcccacctttacgctccgacaagttggaataatatgtgagcgtctcttaaaagactatgaagataaaattcgggaggaatatgagcaaatcctcaatactaaactagcagaacaatatgaatcttttgtgaaattcacacatgatcagattatgcgacgatatgggacaaggccaacaagctatgtatcc

SEQ ID NO: 4 - protein, 192 aa, Bovine
MACGATLKRPMEFEAALLSPGSPKRRRCAPLSGPTPGLRPPDAEPPPLLQTQIPPPTLQQPAPPGSDRRLPTPEQIFQNIKQEYSRYQRWRHLEVVLNQSEACTSESQPHSSTLTAPSSPGSSWMKKDQPTFTLRQVGIICERLLKDYEDKIREEYEQILNTKLAEQYESFVKFTHDQIMRRYGTRPTSYVS

SEQ ID NO: 5 - DNA, 2071 bp, mouse
ccacattcactgtgcaagtcgtggggaaatacagatgaataaaggcttccttgttattctcaaggaatgtatggttttgaagcacagttagacatatattcaaattacagcttcctcctttaaaacactaatattccaaggcacactcaatgttttaaaggatcacagagtgactaccaaagcacgtagcaaaaccctactaagagaggtgtgtttaaaatgactacccaagggacatacttttcaagtcttctaatcgttcactttggatctgtttataccacaagaaaacaatttacttgatgctcttaggtccccttaaaaaataaccatcgtgaagtggcttttcatgtccttggcttttattgaacatagaaacagccatgcaagcggtcttaaaggctttattacatcattgtttcctaataaagtcatgacagtctacctttggaattaaagtgatacacaaaatgatggtctgtgtcctctggtgaactggttccattcagataacacctattcatcatgactatggtttcatttttctttagccttcaagaagctcagaactgaattttaaattcagtcatttaccaccaagataattgtgagttttttttttttaaaaaaactctaatgttttatttctagattttagtttaaaccacgttacatctatattgacaataaatgtgctaaaataaacttaacatgggtaatgtgcctagggaggcttgaatcccaatatggcaaaacaaacagaaaaccagcaatttggtatgctgtgctgtcttatattttacagaaataaatgtgaaagtatatgacctatgttatgatctttaaagagtttgtagaaacggaagaggactcagagaaaagcaaccaaaacgaacaggaggagaaggaagaagaggcggagaaggaggaggaagattggagatagtatgcctttattgtctaaccccaagtgtgttgaagtactgtgacagccatcttggcaattagaaatgagtatctaaaatttggactgttctagaaaaatctgttacagagataatgttaaagccagattacaggaatcacagccactaatatacaaataattacagaaaggctttgaatgtggaggtgttgttctgatgactctattgatgtatttgaaagcactggagttactccccaggaaaattacaaccagagttccctaaagcagaacctccctgttttctattcatttgctgaatatcaaaagcattttccagccaacagtacggcagagaatctcgattgacccgaggaagaaccagtctgagttgccaagtcggatgaggaagccaactgccaaatcagctatcaggggaagttcctaacaccctggtatcacttggttagacagtttaagccagtgagttttctggtaggattgttttttggttttttttttttccttttaatccttttttgcgtaacacatatccatttagtgatccgattaatggccgggtcatctatccccaaaatacattcatttgtaacacacctccccttccaattttgcccatgattgcacagggttcgtggattaaataaagtctatccttagataacccggttatgtttgtgaagatttcctgggactcaagacaaaatcctttgataaccctttagaatcacctcttttatcggtcacgcggccaagggaacccgggtctcccagggtctctcccatcccccgcccccgaggcccctgccgcgcaggtgcgaaagacctcccaggccactccggcagagagcgtgaaggggggggccctgggaggggcgggggcgggggtgttgctaggcgaccacgctctccgcccagaccggcctacttcttccgcagggggcgccatgggccgagcccaggctcgcgggcctcccggatcggcccttttccgacttcttcccctctgccgggcggtggcgcacgcccgtgacgtcacaggaggcggggccagcgcggctgccgggtgccggaggcgccattggagccggcttggcttgggagccgtagctgaagagttggatc

SEQ ID NO: 6 - DNA, 25 bp, Artificial Sequence
  oligonucleotide
caccatggcgtgcggggcgacactg

SEQ ID NO: 7 - DNA, 21 bp, Artificial Sequence
  oligonucleotide
ggatacatagcttgttggcct

SEQ ID NO: 8 - DNA, 20 bp, Artificial Sequence
  oligonucleotide
tgaagcggcccatggagttc

SEQ ID NO: 9 - DNA, 22 bp, Artificial Sequence
  oligonucleotide
ggtgggctggtccttcttcatc

SEQ ID NO: 10 - DNA, 25 bp, Artificial Sequence
  oligonucleotide
agatctgatccaactcttcagctac

SEQ ID NO: 11 - DNA, 24 bp, Artificial Sequence
  oligonucleotide
gctagcccacattcactgtgcaag

(19) United States
(12) Patent Application Publication
(10) Pub. No.: US 2008/0051328 A1
(43) Pub. Date: Feb. 28, 2008

(54) Novel Muscle Growth Regulator

(75) Inventors: Mridula Sharma, Hamilton (NZ); Carole Berry, Hamilton (NZ); Mark Thomas, Hamilton (NZ); Ravi Kambadur, Hamilton (NZ); Robert Syndecombe Bower, Dunedin (NZ)

Correspondence Address: KNOBBE MARTENS OLSON & BEAR LLP, 2040 MAIN STREET, FOURTEENTH FLOOR, IRVINE, CA, 92614, US

(73) Assignee: Orico Limited, Dunedin (NZ)

(21) Appl. No.: 10/580,901

(22) PCT Filed: Nov. 26, 2004
(86) PCT No.: PCT/NZ04/00308

§ 371 Date: May 4, 2007

(30) Foreign Application Priority Data
Nov. 28, 2003 (NZ) 529860

(51) Int. Cl. A61K 38/17; A01K 67/027; C07K 14/435; C12N 15/11; C12N 15/86; G01N 33/53; C12Q 1/68; C12N 15/85; C07K 16/18; A61K 31/7088

(52) U.S. Cl. 514/12; 435/320.1; 435/455; 435/456; 435/6; 435/7.92; 514/44; 530/350; 530/387.9; 530/402; 536/23.1; 800/13; 800/15; 800/16; 800/17; 800/18; 800/19

(57) ABSTRACT

A muscle growth regulating factor includes polynucleotide and polypeptide sequences, promoter sequences, constructs comprising the sequences, and compositions for regulating muscle growth and treating diseases associated with muscle growth. The present sequences can be used in identifying animals with altered muscle mass, and for selective breeding programs to produce animals with altered muscle mass.

### FIELD OF THE INVENTION

This invention relates to a novel protein involved in the regulation of muscle growth and the use of the novel protein in regulating or promoting muscle growth and treating conditions associated with muscle growth or muscle wasting.

### BACKGROUND OF THE INVENTION

Muscle tissue comprises large, multinuclear cells. The bulk of these cells, approximately two thirds, is myofibrils, or the contractile units. Myofibrils are made up of myosin thick filaments and actin thin filaments.

The development of a muscle cell begins with a myoblast or precursor cell. Myoblasts undergo a differentiation and fusion process to form myotubes, which in turn differentiate further to become muscle fibers.

The protein myostatin (or Growth Differentiation Factor 8) has been identified as a major factor in regulating muscle growth and development. Myostatin was shown to negatively regulate muscle growth (Kambadur et. al. 1997). An 11bp deletion in myostatin has been shown to cause the Belgian Blue (or double-muscled) phenotype in cattle. Belgian Blue cattle have a 20% to 30% increase in muscle mass.

The exact mechanism by which myostatin acts to retard muscle growth is still being elucidated.

During periods of prolonged disuse (e.g. bed rest, space flight), or in cases of muscle wasting diseases (e.g. muscular dystrophy) skeletal muscle undergoes atrophy, which is primarily due to enhanced degradation of muscle protein and a reduction in protein synthesis. Duchenne muscular dystrophy is one of the most common forms of muscular dystrophy. Muscle fibres undergo necrosis and lose their ability to regenerate. It has been shown recently that in mdx mice, a duchenne muscular dystrophy model, muscle is unable to regenerate due to an exhaustion of satellite cells rather that fibrosis. Sarcopenia is the decline in muscle mass and performance associated with normal aging. The skeletal muscle is still capable of regenerating itself but it appears that the environment in old aged muscle is less supportive towards muscle satellite cell activation, proliferation and differentiation.

Many growth factors are involved in regulating postnatal skeletal muscle growth and development for example IGF, HGF and FGF. No known growth factor has a more potent negative effect on skeletal muscle development than myostatin (GDF-8). Myostatin or Growth and Differentiation Factor-8 (GDF-8) was first characterised in mice. Myostatin-null mice displayed drastically increased muscle development and weighed 2 to 3 times more than wild-type mice. The increase in muscle mass was shown to be due to both muscle hyperplasia and hypertrophy. These data suggest that myostatin has an important role in controlling muscle mass and that myostatin is a potent negative regulator of muscle growth.

Therefore, it would be beneficial to identify further factors involved in the regulation of muscle growth, including a factor that is able to promote muscle growth. To date, no further factor has been identified which is able to regulate muscle growth.

### STATEMENT OF THE INVENTION

The present invention is based upon the identification of a polypeptide involved in promoting muscle growth. This muscle growth promoter has been termed “mighty”. The term mighty is used throughout this specification to refer to the novel muscle growth promoter according to the present invention.

The present invention is also based on the identification of the DNA that encodes the mighty protein and the corresponding mighty gene promoter.

The present invention provides for a polypeptide comprising a sequence selected from SeQ ID No: 2 or SEQ ID No: 4.

The present invention also provides for a polynucleotide sequence that encodes a polypeptide comprising a sequence selected from SeQ ID No: 2 or SEQ ID No: 4. The polynucleotide sequence includes a sequence selected from SEQ ID No. 1 or SEQ ID No. 3.

The invention also provides for a polynucleotide comprising a nucleotide sequence selected from the group consisting of:

a) complements of SEQ ID No: 1 or SEQ ID No: 3,

b) reverse compliments of SEQ ID No: 1 or SEQ ID No: 3, and

c) reverse sequences of SEQ ID No: 1 or SEQ ID No: 3.

The polynucleotide of the present invention includes a nucleotide sequence that differs from SEQ ID No: 1 or 3 as a result of silent substitution(s) or substitution(s) that results in conservative substitution(s) in the resulting amino acid.

The polypeptide of the present invention includes a polypeptide encoded by a polynucleotide according to the present invention. A fusion protein comprising at least one polypeptide, or a fragment thereof, is also provided for.

The present invention also provides one or more vectors comprising the sequences of the present invention, and one or more host cells containing such vectors. The vector comprises, in the 5′-3′ direction: a) a gene promoter sequence; b) a polynucleotide sequence according to the present invention; and c) a gene termination sequence. The polynucleotide may be in a sense or an anti sense orientation.

The invention also provides a composition for regulating muscle growth.

In one aspect the composition includes any one of:

a) a polynucleotide including SEQ ID No. 1 or SEQ ID No.3,

b) a fragment or variant of (a),

c) a polynucleotide having at least 95%, 90% or 70% sequence identity to (a),

d) a complement of any one of (a) to (c),

e) a reverse complement of any one of (a) to (c),

f) an antisense polynucleotide of any one of (a) to (c),

g) a polypeptide encoded by any one of (a) to (c),

h) a polypeptide including SEQ ID No. 2 or SEQ ID No. 4,

i) a fragment or variant of (g) or (h), and

j) a polypeptide having at least 95%, 90% or 70% sequence relating to (g) or (h).

In another aspect the composition may include the mighty gene promoter including a sequence of SEQ ID No. 5, a polynucleotide having at least 95%, 90% or 70% identity to SEQ ID No. 5, or a fragment or variant thereof.

In a further aspect the composition may include a modulator of mighty gene expression or mighty protein activity.

The modulator of mighty gene expression or mighty protein activity may specifically bind to a polynucleotide selected from any one of:

a) SEQ ID No.1, SEQ ID No. 3, or SEQ ID No. 5,

b) a polynucleotide that encodes a polypeptide of SEQ ID No. 2 or SEQ ID No. 4,

c) a polynucleotide having at least 95%, 90% or 70% sequence identity to (a) or (b),

d) a complement of any one of (a) to (c),

e) a reverse complement of any one of (a) to (c), and

f) a fragment or variant of any one of (a) to (e).

The modulator of mighty gene expression can be an anti-sense polynucleotide. The modulator of mighty gene expression may also be an interfering RNA molecule. Specifically, the modulator of mighty gene expression may be an RNAi or siRNA molecule.

The modulator can also be myostatin or a mimetic of myostatin. The mimetic can be a myostatin peptide C-terminally truncated at or between amino acid positions 330, and 350. The truncation can be at any one of 330, 335 or 350.

In a further aspect, the compositions of the present invention may be used in the treatment or prophylaxis of diseases associated with muscle growth. The disease may be a disease that results in muscle atrophy. The disease may be selected from muscular dystrophy, muscle cachexia, atrophy, hypertrophy, muscle wasting associated cancer or HIV, amyotrophic lateral sclerosis (ALS), or diseases associated with cardiac muscle growth, including infarct. The composition may also be used in promoting muscle regeneration after muscle injury.

The present invention also provides for a method of regulating or promoting muscle growth, the treatment or prophylaxis of diseases associated with muscle growth or muscle regeneration following injury, using a composition according to the present invention. The method may be used to produce an animal having increased muscle mass.

The compositions of the present invention may also be used in the production of a medicament for regulating muscle growth, the treatment or prophylaxis of diseases associated with muscle growth or muscle regeneration following injury.

The invention also provides for a transgenic animal transfected with a composition according to the present invention. The transgenic animal may result in an animal having an increased muscle mass. The transgenic animal may be selected from a sheep, cow, bull, deer, poultry, turkey, pig, horse, mouse, rat, fish or human.

The present invention also provides a method of predicting muscle mass in an animal, including the steps of:
i) obtaining a sample from the animal,
ii) determining the gene expression level from a polynucleotide having a sequence of SEQ ID No. 1 or SEQ ID No.3, a polynucleotide having at least 95%, 90% or 70% sequence identity to SEQ ID No. 1 or SEQ ID No.3, or a fragment or variant thereof; or determining the amount of a polypeptide having a sequence of SEQ ID No.2 or SEQ ID No.4, a polypetide having at least 95%, 90% or 70% sequence identity to SEQ ID No. 2 or SEQ ID No.4, or a fragment or variant thereof,
iii) comparing the gene expression level or amount of polypeptide to an average; and
iv) predicting the muscle mass of said animal.

The level of gene expression may be determined using RTPCR or northern analysis. The polypeptide may be determined using ELISA or Western blot analysis.

In a further aspect the invention provides for a method of detecting a variant of mighty, comprising the use of a nucleotide sequence selected from:

a) SEQ ID No.1, SEQ ID No. 3, or SEQ ID No. 5,

b) a polynucleotide that encodes a polypeptide of SEQ ID No. 2 or SEQ ID No. 4,

c) a polynucleotide having at least 95%, 90% or 700% sequence identity to (a) or (b),

d) a complement of any one of (a) to (c),

e) a reverse complement of any one of (a) to (c), and

f) a fragment or variant of any one of (a) to (e), to screen a sample from an organism for the variant of mighty.

The variant of mighty may be a polymorphism, and in particular a single nucleotide polymorphism. The variant of mighty may also be associated with an altered muscle phenotype.

The invention also provides a method of improving the muscle mass of an animal comprising the steps of:
i) selecting one or more animals predicted to have an increase in muscle mass according to the present invention, and
ii) breeding the one or more animals predicted to have an increased muscle mass to produce an animal having an improved muscle mass.

The animal according to the present invention may be selected from a sheep, cow, bull, deer, poultry, turkey, pig, horse, mouse, rat, fish or human.

The invention also provides for antibodies that preferentially bind a polypeptide having a sequence of SEQ ID NO. 2 or SEQ ID NO. 4 or a polypeptide having at least 95%, 90% or 70% sequence identity to SEQ ID NO. 2 or SEQ ID NO. 4.

The invention also provides for the use of an antigenic fragment of a polypeptide having a sequence of SEQ ID NO. 2 or SEQ ID NO. 4 in the production of an antibody that preferentially binds a sequence of SEQ ID NO. 2 or SEQ ID NO. 4 or a polypeptide having 95%, 90% or 70% identity to SEQ ID NO. 2 or SEQ ID NO. 4.

The present invention also provides an isolated polynucleotide comprising a sequence of SEQ ID No: 5, which comprises the promoter region of the murine mighty gene, a polynucleotide having at least 95%, 90% or 70% sequence identity to SEQ ID No. 5, or a fragment or variant thereof.

The fragment can comprise at least the 200 nucleotides upstream of the mighty initiation site, and may comprise any one of 209, 287, 315, 400, 600, 1000 and 2100 nucleotides upstream of the mighty initiation site.

The present invention also provides one or more vectors comprising a polynucleotide of SEQ ID No: 5, a polynucleotide having at least 95%, 90% or 70% sequence identity to SEQ ID No. 5, or the fragment or variant thereof, and one or more host cells containing such vectors.

The present invention also provides a method of screening for one or more compounds that are potentially useful in inhibiting or promoting muscle growth, including the steps of:
i) inserting a polynucleotide having a sequence of SEQ ID No: 5, a polynucleotide having at least 95%, 90% or 70% sequence identity to SEQ ID No. 5, or a fragment or variant thereof into a suitable vector linked to a suitable marker gene;
ii) transforming a suitable host cell with the vector;
iii) administering a compound of interest to the host cell; and
iv) determining any difference in the level of the marker gene expression.

The vector may include any suitable vector, and may include, a prokaryotic plasmid, a eukaryotic plasmid or a viral vector. The marker gene may include any suitable marker gene, and may include a polynucleotide that encodes a green fluorescent protein, a red fluorescent protein, a luciferase enzyme, or a β-galactosidase enzyme.

The invention also provides a method of expressing a desired protein in a muscle cell, including the steps of:
i) isolating a polynucleotide sequence that encodes the gene to be expressed;
ii) inserting a polynucleotide having a sequence of SEQ ID No: 5, or a polynucleotide having at least 95%, 90% or 70% sequence identity to SEQ ID No. 5, or a fragment or variant thereof, operably linked to the polynucleotide sequence of the protein to be expressed in a 5′-3′ orientation, into a suitable vector, and
iii) introducing the vector into a muscle host cell.

The vector may include a eukaryotic vector, viral vector, or any vector suitable for gene therapy.

The host cell may include a primary myoblast cell line, a transformed myoblast cell line or any cell line in which the mighty promoter is active. The host cell may also include an in vivo skeletal or cardiac muscle cell of a host animal.

The host animal may include a sheep, cow, deer, bull, poultry, turkey, pig, horse, mouse, rat fish or human.

### DEFINITIONS

The term “polynucleotide” is to be understood as meaning a polymer of deoxyribonucleic acids or ribonucleic acids, and includes both single stranded and double stranded polymers, including DNA, RNA, cDNA, genomic DNA, recombinant DNA and all other known forms of polynucleotides. The polynucleotide may be isolated from a naturally occurring source, produced using recombinant or molecular biological techniques, or produced synthetically. A polynucleotide may include a whole gene or any part thereof, and does not have to have an open reading frame.

The use of all polynucleotides according to the present invention includes any and all open reading frames. Open reading frames can be established using known techniques in the art. These techniques include the analysis of the sequences to identify known start and stop codons. Many computer software programmes that can perform this function are known in the art.

The term “polypeptide” is to be understood as meaning a polymer of covalently linked amino acids. A polypeptide includes a polypeptide that has been isolated from a naturally occurring source, a polypeptide that has been produced using recombinant techniques, or a polypeptide that has been produced synthetically. It is to be appreciated that a polypeptide that includes a leader or pro-sequence which is cleaved off in, vitro, or a polypeptide that includes a linker or any other sequence, or a polypeptide that undergoes a post-translational modification is intended to come within the definition of polypeptide.

The term “fragment or variant” is to be understood to mean any partial sequence or sequence that has been modified by substitution, insertion or deletion of one or more nucleotides or one or more amino acid residues, but has substantially the same activity thereof.

A polynucleotide fragment also includes a polynucleotide fragment of sufficient length and specificity to hybridise under stringent conditions to a sequence of SEQ ID No: I of SEQ ID No: 3. An example of “stringent conditions” involves pre-hybridisation with 5×SSC, 0.2% SDS at 65° C.; performing the hybridisation overnight in 5×SSC, 0.2% SDS at 65° C.; two washes of 1×SSC, 0.1% SDS at 65° C. for 30 min each; followed by a further two washes of 0.2×SSC, 0.1% SDS at 65° C., also for 30 min each.

A polypeptide fragment also includes a fragment that retains the activity of the mighty protein. This fragment may have enhanced activity and therefore, when introduced or expressed in a cell, results in an increase in mighty protein activity. Alternatively, the fragment may have a dominant negative effect.

“Mighty gene” is defined as a polynucleotide according to SEQ ID No. 1 or SEQ ID No. 3, or a polynucleotide having 95%, 90% or 70% identity to SEQ ID No. 1 or SEQ ID No. 3 or a fragment thereof.

“Gene expression” is defined as the initiation of transcription, the transcription of the mighty gene into mRNA, and the translation of the mRNA into a polypeptide. “A modulator of mighty gene expression” is defined as any compound that is able to cause an increase or a decrease in mighty gene expression.

“Mighty protein” is defined as a polypeptide having a sequence of SEQ ID No. 2 or SEQ ID No. 4, a polypeptide having 95%, 90% or 70% identity to SEQ ID No. 2 or SEQ ID No. 4, or a fragment or variant thereof.

“Mighty protein activity” is defined as the ability of the mighty protein to stimulate muscle growth.

“Muscle growth” is defined as the division and/or differentiation of muscle cells and includes the division and/or differentiation of any muscle precursor cell.

“A modulator of mighty protein activity” is defined as a compound that is able to increase or decrease mighty protein activity.

The “mighty gene promoter” is defined as a polynucleotide of SEQ ID No. 5, a polynucleotide having 95%, 90% or 70% identity to SEQ ID No. 5, or a fragment or variant thereof.

Further aspects of the present invention will become apparent from the following Figures and description, given by way of example only.

### BRIEF DESCRIPTION OF THE FIGURES

The invention will now be described by way of example only with reference to the following figures:

FIG. 1: Shows the PCR amplification of mighty from double muscled cattle and normal muscled cattle.

FIG. 2: (A) shows the PCR amplification of mighty from the heart tissue of a normal muscled cattle (wt, lane 1) and a double muscled phenotype (BB, lane 2). (B) shows the PCR amplification of mighty from ovine skeletal muscle (lane 4).

FIG. 3: (A) and (B) shows the mighty promoter sequence, and the identified transcription factor binding sites.

FIG. 4: Shows the results of expression of mighty in myoblast C2C12 cell proliferation.

FIG. 5: Shows immunostaining of control and mighty over-expressing myotubes with MHC antibody.

FIG. 6: Shows the measurement of actively growing C2C12 clones 7 and 11 and the lacZ control, measured by (A) quantitative image analysis of cell area. (B) FACScan flow cytometry measuring forward angle light scatter (FALS) (The shift to the right seen in clone 7 and clone 11 indicates an increase in cell size) and length (C), width (D) and area (E) of 3 nuclei containing myotubes, measured by quantitative image analysis.

FIG. 7: Shows: (A) mighty overexpressing C2C12 clones 7 and 11, and control C2C12 myoblasts were cultured in differentiation media (DMEM 2% HS) for 48, 60 and 72 hours. Myoblasts were fixed and immunostained using anti-MHC antibodies, and lightly counterstained with Gills haematoxylin. (B) Western blot analyses of mighty overexpressing C2C12 clones 7 and 11, and control C2C12. The myoblasts were cultured in differentiation media (DMEM 2% HS) for 0, 24, 48 and 72 hours. Total protein (15 μg) extracted from cells were resolved by 4-12% SDS-PAGE, transferred to nitrocellulose filters, and probed with mouse monoclonal anti-p21, rabbit polyclonal anti-MyoD or mouse monoclonal anti-MHC antibodies. Filters were also probed with mouse monoclonal anti-α-tubulin antibody to demonstrate equal loading.

FIG. 8: Shows the detection of mighty protein in quiescent and activated satellite cells. Protein from quiescent and activated satellite cells was resolved by SDS-PAGE and transferred to nitrocellulose membrane. Mighty protein was detected with rabbit anti-mighty antibody. Mighty protein was detectable in activated satellite cells but not in quiescent satellite cells.

FIG. 9: Shows the expression of mighty during skeletal muscle regeneration; TA muscle was injected with Notexin to induce muscle injury and collected for immunohistochemistry of mighty on days 0, 1, 5, 14, and 28 post-injury. (A) Non-injured control TA muscle on day 0. (B) Day 1 post-injury. (C) Day 5 post-injury. (D) Day 7 post-injury. (G) Day 28 post-injury. Green, Mighty; Blue, DNA.

FIG. 10: Shows the expression of mighty in heart tissue post-infarction. Immunohistochemistry for mighty was performed on non-infarcted (day 0) and post-infarction (day 2 and 6) heart tissue. (A) Non-infarcted control heart tissue. (B) Day 2 post-infarction. (C) Day 6 post-infarction.

FIG. 11: Shows the number of MHC positive myotubes in mighty and control transfected human myoblasts. Human myoblasts were transfected with mighty-pcDNA3 or pcDNA3 only (control), and cultured under differentiating conditions for 12 h. Immunohistochemistry (ICC) was then performed for myosin heavy chain (MHC) expression in myotubes. Sequential non-overlapping photographs were taken at intervals across the entire well and the number of MHC positive myotubes/well was counted.

FIG. 12: Shows (A) the frequency of myonuclei per myotube and (B) the widths of myotubes containing 5, 6, 7, 8, and 9 myonuclei. Human myoblasts were transfected with mighty-pcDNA3 or pcDNA3 only (control) and cultured under differentiating conditions for 12 h, and ICC for MHC expression performed. MHC positive myotubes containing 5, 6, 7, 8, and 9 myonuclei (n=53) were measured at their widest width.

FIG. 13: Shows the number of myonuclei/MHC positive myotube in conditioned media treated human myoblasts. Human myoblasts were treated with conditioned media from mighty stably transfected C2C12 cells or LacZ (control) transfected C2C12 cells. Cells were cultured with conditioned media for 48 h then ICC performed for MHC expression. The number of myonuclei/MHC expressing myotube were counted in 5 myotubes per microscopic field (n=245 myotubes). (A) Number of myotubes with 1-3, 4-6, and 7-9 myonuclei. (B) Average number of myonuclei/myotube.

FIG. 14: Shows the widths of conditioned media treated myotubes containing 8 myonuclei. Human myoblasts were treated with conditioned media from mighty stably transfected C2C12 cells or LacZ (control) transfected C2C12 cells. Cells were cultured with conditioned media for 48 h then ICC performed for MHC expression. MHC positive myotubes containing 8 myonuclei (n=100) were measured at their widest width.

FIG. 15: Shows the number of MHC positive myotubes in conditioned media treated human Rhabdomyosarcoma (RD) cells. Human RD cells were treated with conditioned media from mighty stably transfected C2C12 cells or LacZ (control) transfected C2C12 cells. Cells were cultured with conditioned media for 72 h then ICC performed for MHC expression. The number of MHC positive myotubes/well was counted.

FIG. 16: Shows the murine mighty promoter has activity in a variety of cell lines. (A) C2C12 myoblasts, primary ovine myoblasts, NIH3T3 fibroblasts, Chinese Hamster Ovary (CHO) and (B) human RD (Rhabdomyosarcoma) cells were transiently transfected with the 1 kb mighty promoter-reporter construct for 24 hours. These were then cultured for a further 24 hours in growth or differentiation media. Luciferase activity was determined and normalised to β-galactosidase (β-gal) activity from cotransfected pCH110.

FIG. 17: Shows that mighty promoter is dose dependently inhibited by myostatin. C2C12 myoblasts were transiently transfected with 1 kb promoter as described in methods. Twenty four hours after the transfection, the cells were treated with 4 and 8 μg/ml of recombinant myostatin in growth media. The cells were harvested after 24 h of the treatment. The luciferase activity was normalised to β-galactosidase activity.

FIG. 18: Shows that myostatin mimetics can restore the myostatin mediated inhibition of the mighty promoter. X-axis is the relative luciferase activity of mighty promoter normalised to β-galactosidase activity. Ctrl bar represents control ovine myoblasts; wt bar represents the ovine myoblasts treated with wild type myostatin (3 μg); 335 bar represents ovine myoblasts treated with 15 μg of myostatin mimetic 335; 335+wt represents ovine myoblasts treated with 3 μg of myostatin and 15 μg of myostatin mimetic.

FIG. 19. Shows the promoter activity of upstream fragments of the mighty gene. C2C12 myoblasts were transiently transfected with the promoter fragment reporter construct as described in methods. These were then cultured for a further 24 hours in growth or differentiation media. Luciferase activity was determined and normalised to β-galactosidase (β-gal) activity from cotransfected pCH110.

FIG. 20: Shows the application of antibodies against the mighty protein. The lanes show: M, Markers; 1, Purified recombinant mouse mighty protein recognised by a peptide specific mighty antibody; 2, Purified recombinant bovine mighty protein recognised by bovine protein antibody; 3, Protein extract from E. coli cells containing mighty expression plasmid induced for mighty expression (bovine protein antibody used); and 4, Protein extract from E. coli cells containing mighty expression plasmid (Uninduced) (bovine protein antibody used).

### DETAILED DESCRIPTION OF THE INVENTION

The present invention is based on a novel protein that is involved in the development and regeneration of muscle. The protein has been termed “mighty”.

The mighty gene has been identified and cloned from both ovine (SEQ ID No:1) and bovine (SEQ ID No:3). In one aspect, the present invention provides for the mighty polynucleotide isolated from bovine and ovine. Specifically, the invention provides a polynucleotide sequence from ovine, SEQ ID No. 1, and bovine SEQ ID No. 3 including anti-sense polynucleotides and operable anti-sense fragments.

The present invention also provides for a polypeptide sequence isolated from bovine and ovine. Specifically, the invention provides a polypeptide from ovine, SEQ. ID No. 2 and bovine SEQ ID No. 4, and polynucleotides that encode the polypeptides of SEQ ID No:2 and SEQ ID No: 4.

It will be appreciated that the polynucleotides, as a result of the redundancy in the genetic code, can include silent substitution(s) or substitution(s) that result in conservative substitution(s) in the resulting amino acid. Furthermore, fragments and variants of the polynucleotides and polypeptides of the present invention that do not substantially alter the activity of the protein are also contemplated.

The invention also provides for vectors containing polynucleotides of the present invention. The use of vectors to store, replicate and express polynucleotide sequences are well known in the art. Generally vectors comprise, in the 5′-3′ direction: a gene promoter sequence, the polynucleotide sequence according to the present invention, and a gene termination sequence. Vectors are intended to include the incorporation of a sequence according to the present invention into a plasmid and/or virus to aid in the introduction and/or maintenance of the sequence in a host cell. The host cell may include, either, a prokaryotic or a eukaryotic cell. The eukaryotic cell may be in vivo, or may be a primary or transformed cell line.

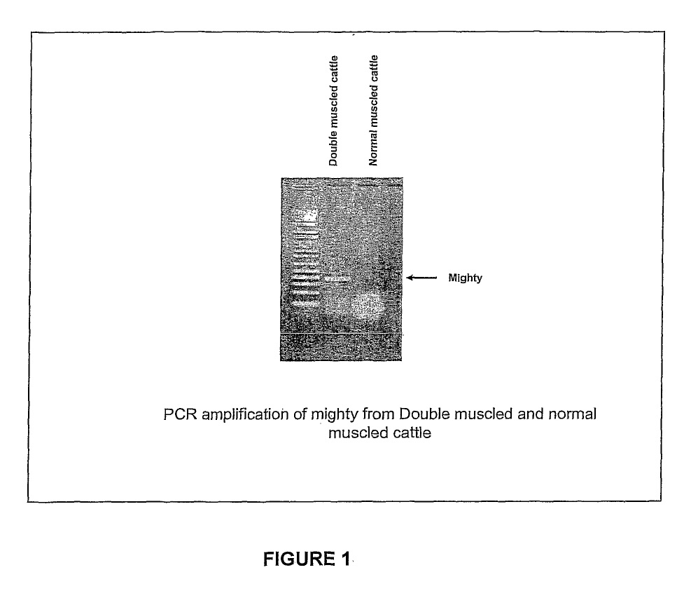

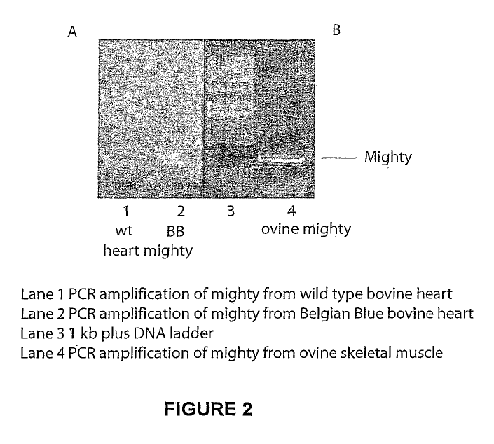

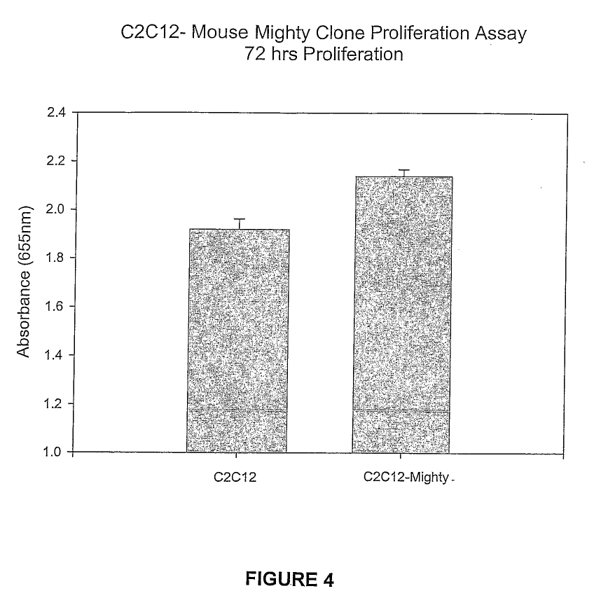

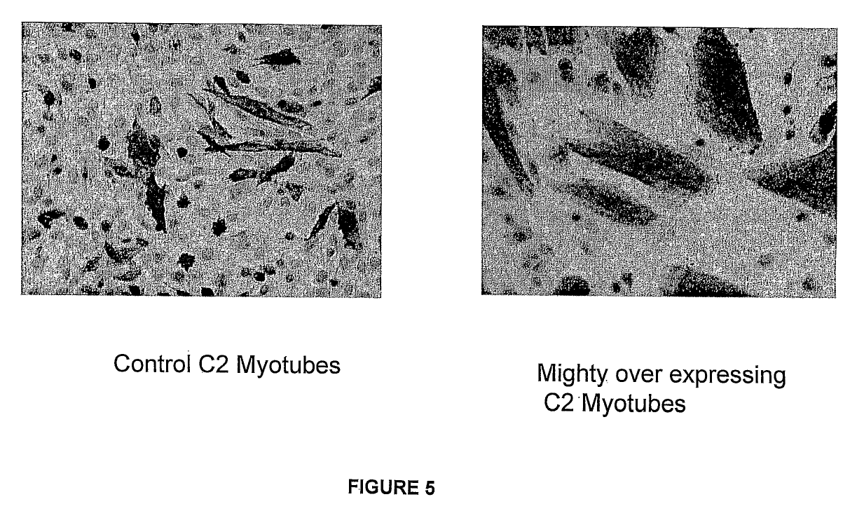

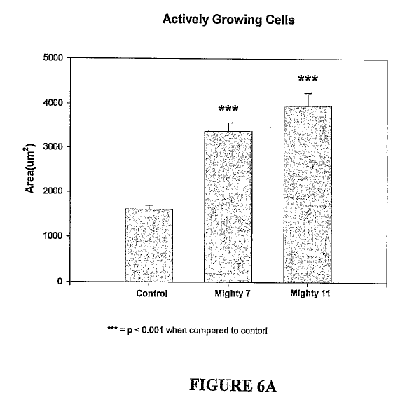

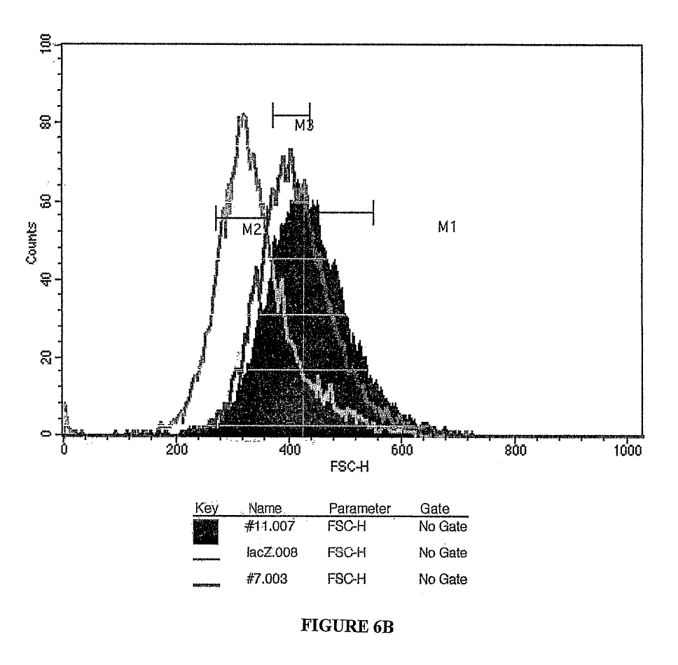

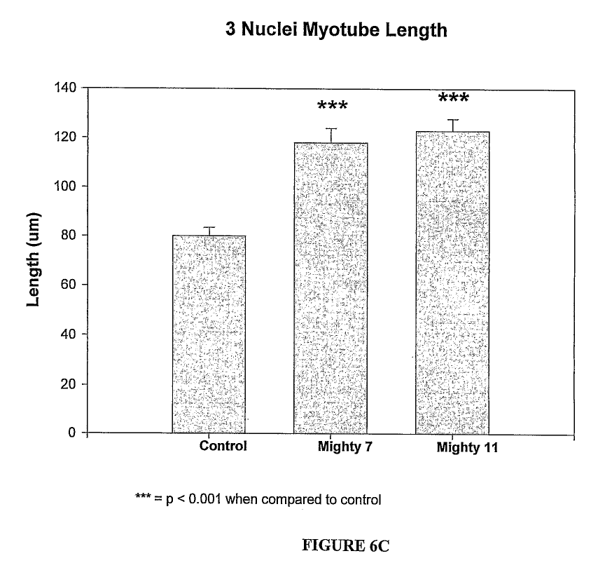

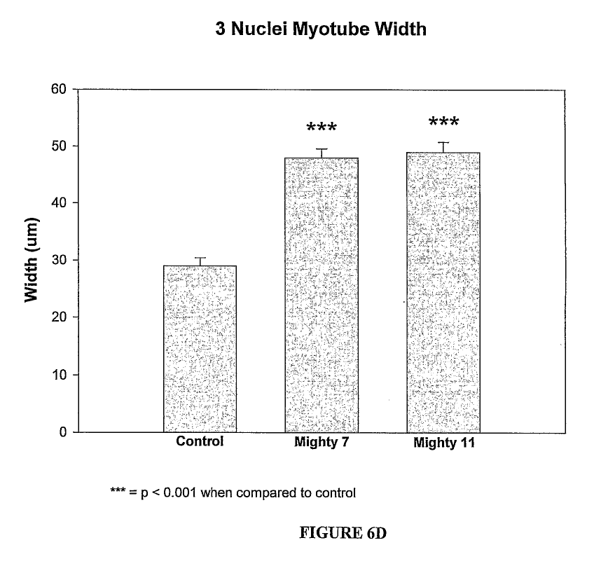

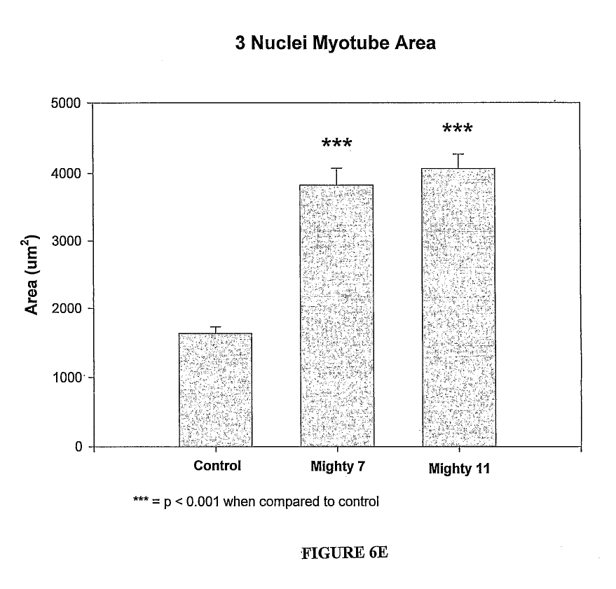

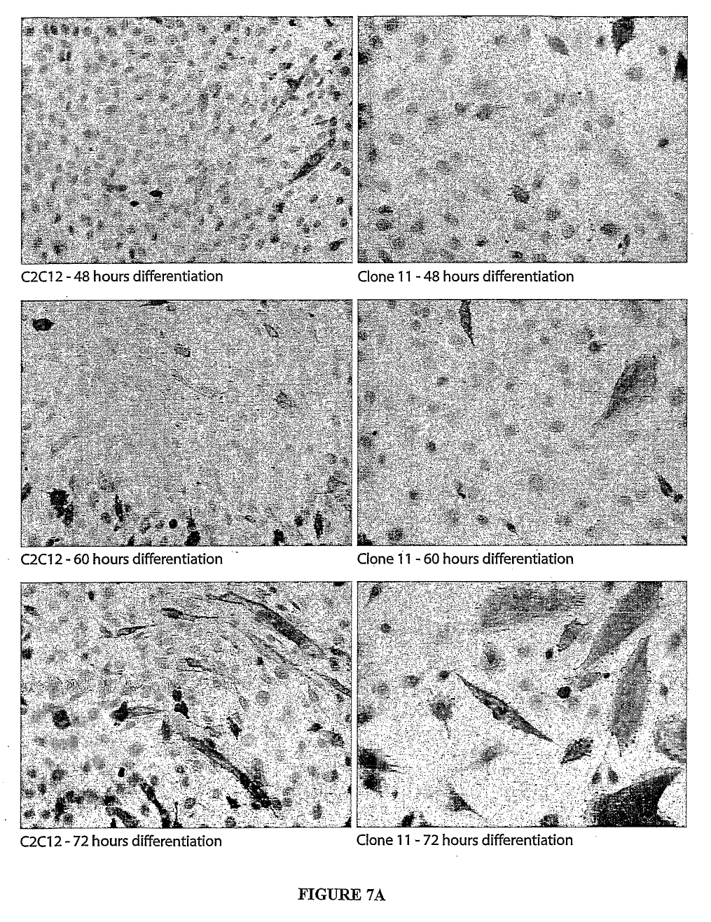

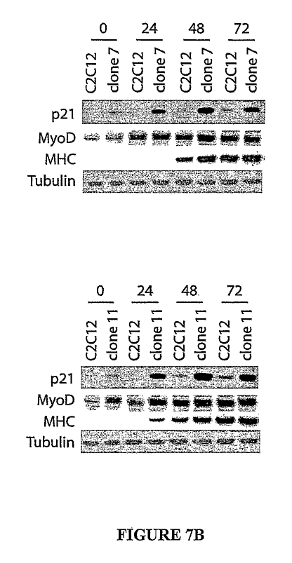

The mighty protein has been shown by the inventors to be highly expressed in doubled muscled cattle (see FIG. 1 and FIG. 2) indicating that mighty plays a role in promoting muscle growth. Mighty has also been shown to up regulate the growth of myoblast C2C12 cells, confirming mighty's role in promoting muscle growth (FIG. 4). Further investigation of C2C12 cells overexpressing mighty show that mighty induces hypertrophy in muscle cells. This is shown by an increase in nuclei number (FIG. 5), and an increase in cell size (FIG. 6) in the cells overexpressing mighty. Furthermore, as shown in FIG. 7, C2C12 cells overexpressing mighty differentiate earlier than control cells. Primary myoblasts (activated satellite cells) have also been shown to have greater levels of mighty than satellite (quiescent cells).

These results confirm the role of mighty in the growth and development of muscle, and shows that mighty could be used to regulate or promote muscle growth and development. Mighty provides a useful tool for producing animals having increased muscle mass that would have agricultural benefits. Furthermore, mighty provides for the development of compositions for treating diseases associated with muscle growth and in particular diseases associated with muscle atrophy. Such diseases include, but are not limited to; muscular dystrophy, muscle cachexia, atrophy, hypertrophy, muscle wasting associated cancer or HIV, amyotrophic lateral sclerosis (ALS), or diseases associated with cardiac muscle growth, including infarct.

Compositions for regulating muscle growth are therefore included. The “regulation of muscle growth” is intended to include any change in the rate of muscle growth and/or development and includes the growth and/or differentiation of any muscle precursor cell. This includes any change in the rate at which precursor muscle cells divide, and/or any change in the rate at which precursor muscle cells differentiate. The change may be either an increase or a decrease.

Such compositions are based on the mighty gene sequences, including the sequences from ovine SEQ ID No. 1, or bovine SEQ ID No. 3, a polypeptide having at least 95%, 90% or 75% sequence identity to SEQ ID No. 1 or SEQ ID No. 5, or a fragment or variant thereof. The sequence may be introduced into a cell by incorporation into a suitable vector under the regulation of a promoter, either the mighty promoter (SEQ ID No: 5), or any other suitable promoter. The promoter may be used to cause expression of the mighty protein, thereby both increasing gene expression and mighty protein activity within the cell.

The composition may also include a sequence having at least 95%, 90% or 70% sequence identity to the polynucleotide sequences of the present invention. Sequence identity may be determined by aligning the sequences and determining the number of identical nucleotides. Many computer algorithms are known for determining sequence identity, for example, the BLASTN algorithm.

The composition may also include complements, reverse complements, or anti-sense polynucleotides of the polynucleotides according to the present invention.

The composition may also comprise a mighty polypeptide according to the present invention. The polypeptide may be from ovine, SEQ ID No. 2, or bovine, SEQ ID No. 4, polypeptides having at least 95%, 90% or 70% sequence identity to SEQ ID No. 2 or SEQ ID No. 4, or fragments or variants thereof.

The composition may also comprise a sequence having at least 95%, 90% or 70% sequence identity to the polypeptide sequences of the present invention. Sequence identity may be determined by aligning the sequences and determining the number of identical residues. Many computer algorithms are known in the art for determining the sequence identity, for example BLASTP algorithm.

The composition for regulating muscle growth may also comprise a modulator of mighty gene expression.

The composition for regulating muscle growth may also include a modulator of mighty protein activity.

A modulator of mighty gene expression may be a compound that can specifically bind to a polynucleotide according to the present invention. Specifically, such a modulator of mighty gene expression could bind to the mighty gene promoter, thereby affecting the rate at which gene transcription is initiated or maintained. Alternatively, the promoter or a fragment thereof could be used to introduce specific alterations into the native promoter of a cell to either enhance or repress wild type mighty gene expression. Alterations can include substitutions, inserts, or deletions of one or more nucleotides.

Another modulator of mighty gene expression may also bind to the mighty gene directly affecting the rate at which the gene is expressed.

Another modulator of mighty gene expression may also operate by binding to the mighty gene and introducing alterations into the sequence, for example, by homologous recombination, which may either affect the rate at which the gene is expressed, or may alter the mighty protein activity. Alterations of a sequence include a nucleotide change, insertion or deletion, which may or may not result in an amino acid change, insertion or deletion in the resulting polypeptide. Examples of alterations can include the insertion of termination codons such that a truncated polypeptide is produced, or the alteration of one or more codons such that one or more amino acid residues are altered. Alternatively, the variations could be to delete a section of the wild type mighty gene, or introduce a section into the mighty wild type gene. Techniques are well known in the art to make such alterations. Furthermore, it would be within the skill of a person skilled in the art to introduce such changes into the mighty gene and then test the alterations on mighty activity, for example using the myoblast proliferation assay as described in example 4.

The mighty gene expression may also be altered by introducing polynucleotides that interfere with transcription and/or translation. For example anti-sense polynucleotides could be introduced, which may include; an anti-sense expression vector, anti-sense oligodeoxyribonucleotides, anti-sense phosphorothioate oligodeoxyribonucleotides, anti-sense oligoribonucleotides, anti-sense phosphorothioate oligonucleotides, or any other means that is known in the art, which includes the use of chemical modifications to enhance the efficiency of anti-sense polynucleotides.

It will be appreciated that any anti-sense polypeptide need not be 100% complementary to the polynucleotides in question, but only needs to have sufficient identity to allow the anti-sense polynucleotide to bind to the gene, or mRNA to disrupt gene expression, without substantially disrupting the expression of other genes. It will also be understood that polynucleotides that are complementary to the gene, including 5′ untranslated regions may also be used to disrupt translation of the mighty protein. Likewise, these complementary polynucleotides need not be 100% complementary, but be sufficient to bind the mRNA and disrupt translation, without substantially disrupting the translation of other genes.

The modulation of gene expression may also comprise the use of an interfering RNA molecule as is known in the art, and include RNA interference (RNAi) and small interfering RNA (siRNA).

Modulation of gene expression may also be achieved by the use of catalytic RNA molecules or ribozymes. It is known in the art that such ribozymes can be designed to pair with a specifically targeted RNA molecule. The ribozymes bind to and cleave the targeted RNA.

Any other technique known in the art of regulating gene expression can also be used to regulate mighty gene expression.

The composition may also include a modulator of mighty activity. A modulator of mighty may include a dominant negative mutant of the mighty protein. A dominant negative effect arises where a mutant acts to block the physiological activity of a wild type protein. This may occur by the dominant negative protein binding to, but not activating, a receptor, while also preventing the wild type protein from binding. Alternatively the dominant negative may act by binding directly to, and inactivating, the wild type protein.

Thus the polynucleotides of the present invention can be used to make suitable compositions, or be used to design suitable compositions that regulate the mighty gene expression, and thereby regulate muscle growth. Such techniques could be used to regulate mighty gene expression within a cell, for example within a primary or transformed cell line, or to regulate muscle growth within an animal.

One possible application of the compositions of the present invention is to promote or inhibit muscle cell growth and/or differentiation. The muscle cell can be either a primary or transformed cell line, or the cell can be an in vivo cell of a host animal. Suitable host animals may include sheep, cows, bulls, deer, poultry, pigs, fish, horses, mice, rats or humans.

The compositions of the present invention may also be used for the treatment of diseases associated with muscle tissue. Such diseases or injury may include muscular dystrophy, muscle ataxia, or diseases associated with cardiac muscle growth. Similarly the compositions may also be used to promote muscle regeneration after muscle injury.

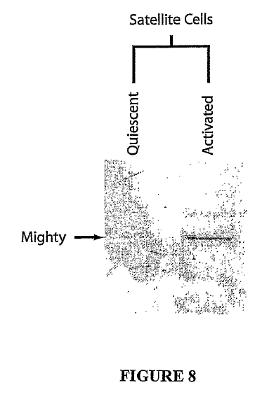

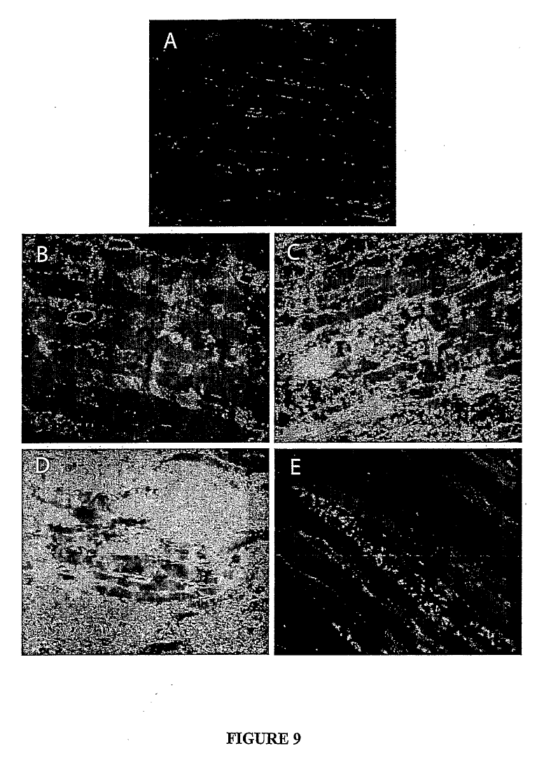

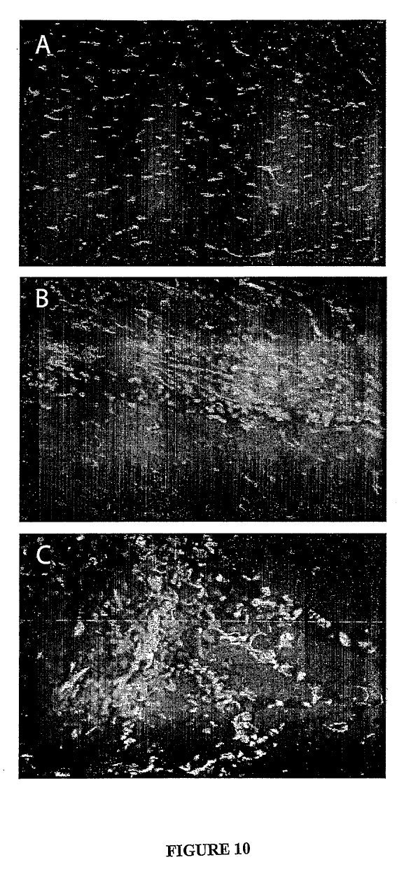

As shown in FIG. 9, mighty expression increases in cells following muscle damage, during muscle regeneration. Furthermore, mighty expression has been shown to increase post infarction in heart tissue in sheep (FIG. 10). These results show that mighty is also involved in muscle regeneration. Therefore, not only are the compositions useful in treating diseases associated with muscle growth, but are also useful in treating muscle damage following injury.

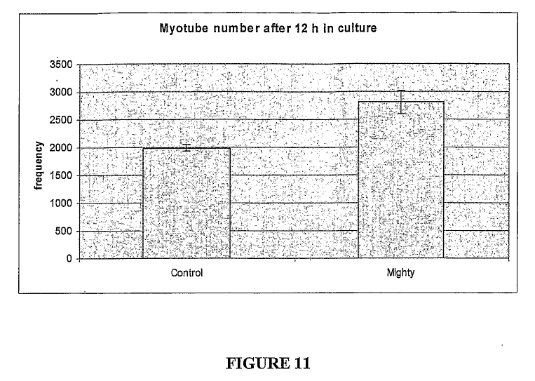

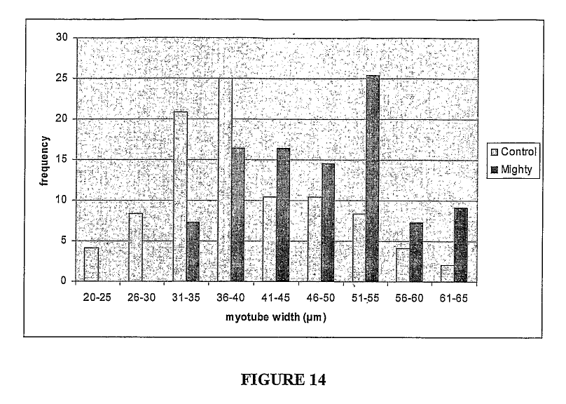

The results in FIGS. 11 to 15 show that the compositions of the present invention can also be used to stimulate growth and differentiation of human muscles, further confirming the potential for human medical applications of the present invention.

Similarly the compositions could be used to produce transgenic animals. The compositions could be used to produce transgenic animals having an increase in muscle mass. Suitable animals may include sheep, cows, bulls, deer, poultry, pigs, fish, horses, mice, rats or humans. Many techniques are known in the art for producing transgenic animals, and any suitable method could be used.

Another application of the present invention may be to predict the muscle mass of an animal. To do this a sample is obtained from an animal. The sample is then analysed for the level of mighty gene expression, or mighty protein. Many techniques are known in the art for measuring gene expression or protein amount. For example, gene expression can be analysed using quantitative RTPCR or northern analysis. Protein content can be determined using ELISA [Enzyme-linked Immunosorbant Assay] or Western blot analysis.

The level of mighty gene expression, or amount of the mighty protein, is then compared to an average. An average level of mighty gene expression is the average level obtained from a sample of animals of average muscle mass. Similarly, the average amount of mighty protein is the amount of protein observed in a sample of animals of average muscle mass.

An increased level of mighty gene expression or mighty protein, compared to the average, means that the muscle mass of the animal will be predicted to have an above average muscle mass. A decreased level of mighty gene expression or mighty protein, compared to the average, means that the muscle mass will be predicted to be less than average.

It will be appreciated that naturally occurring variants of the mighty gene may exist. Such variants may include polymorphisms, for example, single nucleotide polymorphisms (SNPs). A person skilled art would be able to use the sequences of the present invention to screen for such variants. For example, the sequences could be used to design suitable primers for use in polymerase chain reaction (PCR) to amplify the mighty gene, or fragments of the mighty gene to screen for such variants in various organisms. The screening could involve techniques such as direct sequencing or single stranded conformational polymorphism analysis (SSCP). It will also be appreciated that variants of mighty may be associated with altered muscle mass of an organism.

The method described above for determining levels of mighty expression or detecting variants of mighty associated with altered muscle mass may be used to pick animals to be involved in a breeding programme to produce offspring with increased or decreased muscle mass.

The invention also provides for antibodies against the mighty protein. Given the sequences disclosed in the present specification, a person skilled in the art would be able to produce antibodies against the mighty protein. Examples of how antibodies can be produced including the production of hybridoma cells can be found in Eryl Liddell and Cryer (1996) or Javois (1999). It will also be appreciate that the binding domain of an antibody is considered to fall within the definition of an antibody.

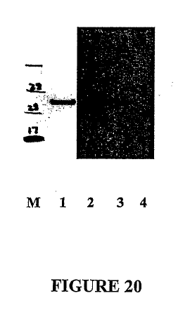

As outlined in example 16 and shown in FIG. 20, polyclonal antibodies can be raised against the entire mighty protein in a suitable animal. Alternatively, antigenic fragments can used to generate peptide specific. This shows how antibodies according to the present invention can be generated using techniques known in the art.

Such antibodies could be used to detect, and/or quantitate mighty protein in a sample. Alternatively, the antibodies according to the present invention could be used to bind and regulate mighty activity.

It will be appreciated that other types of antibodies and binding proteins can be produced and are contemplated to be part of the present invention. These include, but is not limited to, non-mammalian antibodies, for example the IgNAR class of antibodies from sharks; bacterial immunity proteins, for example a IMM7 immunity protein from E. coli, or any other class of binding protein known in the art. Given the sequences disclosed in the present specification, a person skilled in the art would be able to produce such a polypeptide or screen a library of known binding polypeptides to obtain a polypeptide that preferentially binds to a polypeptide of the present invention.

The mouse mighty promoter has also been isolated and cloned (SEQ ID No:5), and also forms part of the present invention. The present invention also provides one or more polynucleotides comprising the mouse mighty promoter. The mighty promoter is a polynucleotide of SEQ ID No: 5, a polynucleotide having at least 95%, 90% or 70% sequence identity to SEQ ID No. 5, or fragments or derivatives thereof.

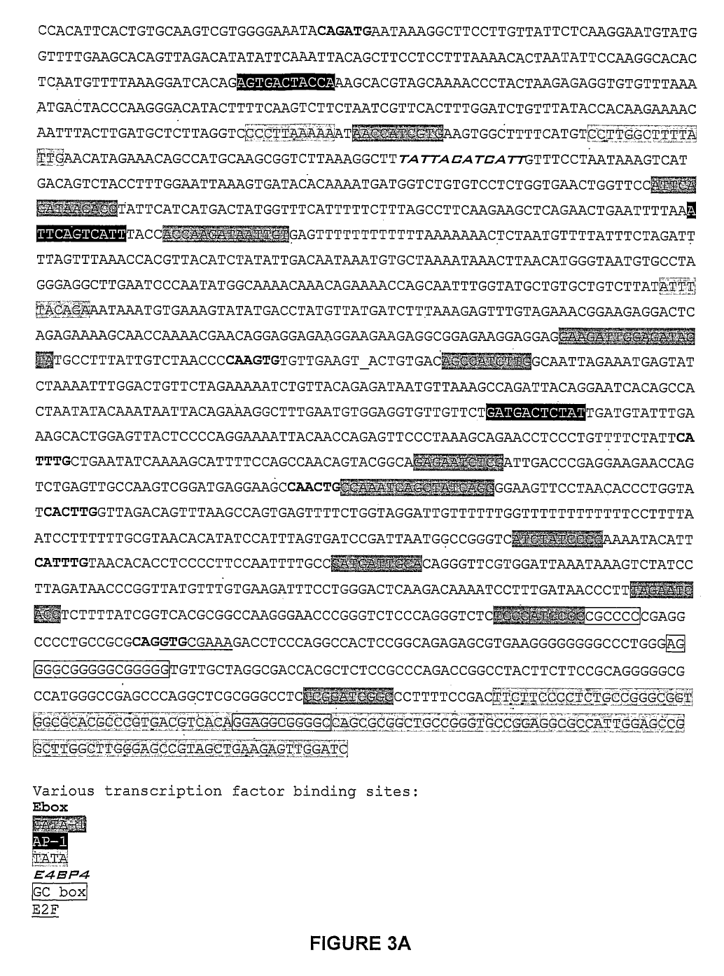

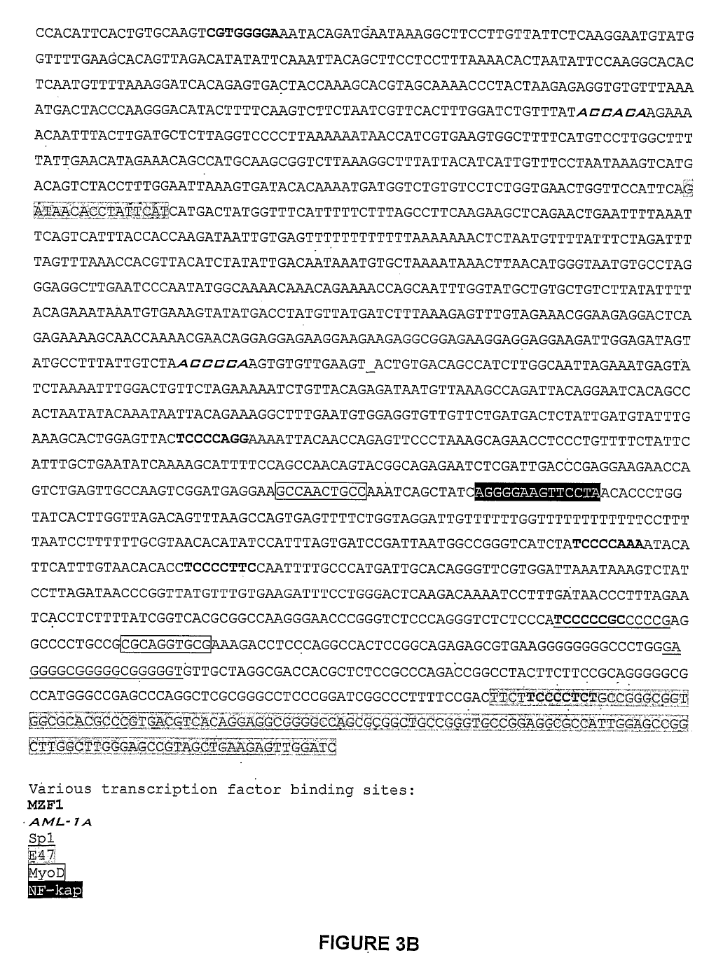

Analysis of the promoter sequence, FIG. 3, shows known transcription factor binding sites.

Vectors containing the mighty gene promoter can be produced using known techniques. Vectors are intended to include the incorporation of the polynucleotide into a plasmid and/or virus to aid the introduction and/or maintenance of the polynucleotide in a host cell. The host cell may be a prokaryotic cell or a eukaryotic cell, an in vivo or a primary or transformed cell line.

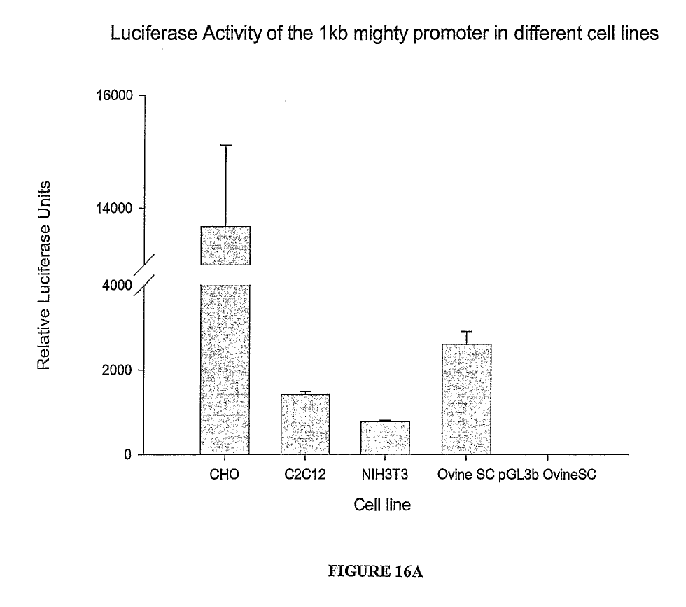

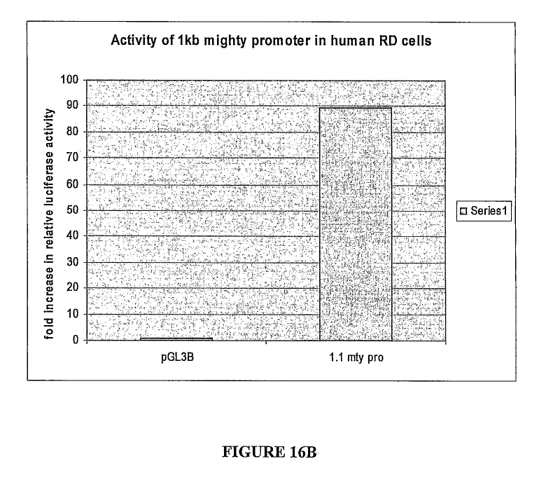

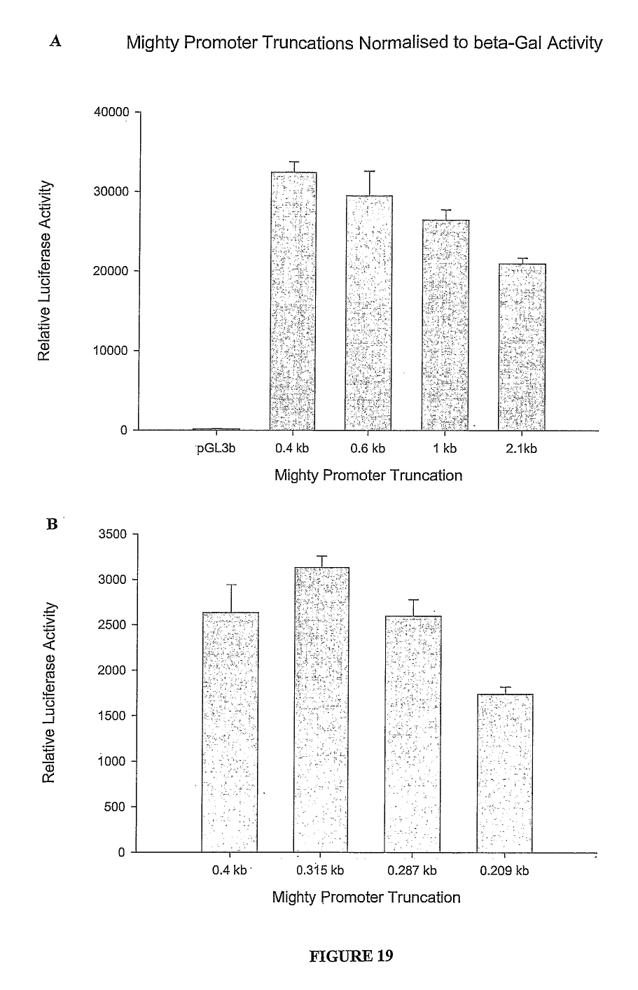

As shown in FIG. 16, the mighty promoter can be used to drive the expression of a gene in various cell lines including human. Furthermore, as shown in FIG. 19, fragments as small as the 200 nucleotides upstream of the mighty initiation site are capable of driving gene expression.

The mighty gene promoter can be used to screen for compounds that may be useful in regulating mighty gene expression, and therefore could be useful in regulating muscle growth. To do this, the mighty promoter can be placed into a suitable expression vector with a suitable marker gene. A “marker gene” is a gene whose expression product may be identified and quantified. Many suitable marker genes are known and may include, for example, green fluorescent protein, red fluorescent protein, luciferase, or β-galactosidase.

The vector is then placed into a suitable host cell using known transfection techniques. A suitable host cell comprises a cell in which the mighty gene promoter is activated, causing the marker gene to be expressed, and levels of the marker gene expression product can be detected. The compound of interest is then applied to the host cell, and any changes in the marker gene determined.

An increase in the amount of the marker gene expression product compared to the base line indicates that the compound may be enhancing gene expression via the mighty gene promoter and therefore may be useful in promoting muscle growth. A decrease in the amount of the marker gene product compared to the base line indicates the compound may be inhibiting gene expression via the mighty gene promoter and therefore may be useful in inhibiting muscle growth.

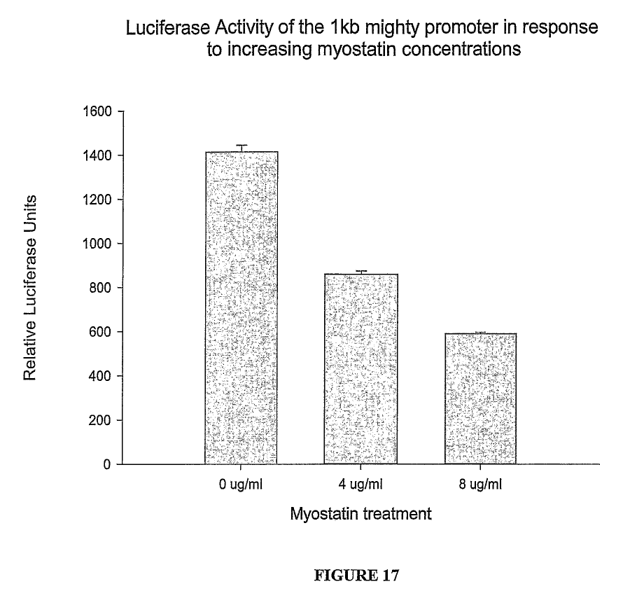

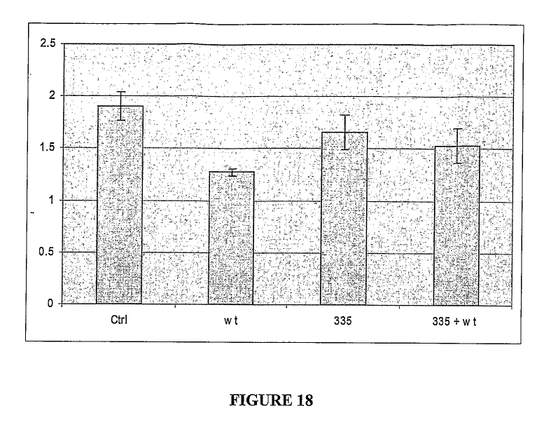

As shown in FIG. 17, this method can be used to show that the mighty promoter is regulated by myostatin. Because myostatin is a known negative regulator of muscle, this result further confirms mighty's activity in promoting and regulating muscle growth and development. Dominant negative mimetics of myostatin are also known. WO 01/53350, C-terminally truncated (between positions 330 and 350) myostatin peptides results in a myostatin mimetic that has a dominant negative effect. As shown in FIG. 18, the myostatin mimetic 335 (truncated at position 335) is able to rescue the myostatin mediated inhibition of the mighty promoter. Combined, FIG. 17 and FIG. 18 shown that both myostatin and myostatin mimetics can be used to down regulate or up regulate mighty expression respectively, and therefore can be used in a composition according to the present invention.

The mighty gene promoter may also be used to express a designated gene in a muscle cell. The designated gene may comprise a polynucleotide according the present invention, or could be any other polynucleotide, for example, a polynucleotide that encodes the myostatin protein. To achieve this, the mighty gene promoter is inserted into a suitable vector in conjunction with the gene of interest. Many suitable vectors are known in the art and may include eukaryotic vectors, viral vectors or any vector suitable for gene therapy.

The vector can then be introduced into a suitable host cell using known transfection techniques.

A suitable host cell can be any muscle cell or mammalian cell where the mighty promoter is activated. The host cell may include, for example, a primary or myoblast cell line, or a transformed myoblast cell line, or a skeletal or cardiac muscle cell of a host animal.

Any host animal where the mighty promoter is active may be used, but may include for example sheep, cows, bulls, deer, poultry, turkey, pigs, fish, horses, mice, rats or humans.

### EXAMPLE 1

### Isolation of Mighty cDNA

RNA Purification: RNA was purified from ovine and bovine skeletal muscle and heart tissue samples using TRIZOL (Invitrogen) according to the manufacturer's protocol.

Amplification of the mighty cDNA: Amplification of mighty cDNA was carried out in a combined reverse transcription PCR. First strand cDNA was synthesized in a 20 μl reverse transcription reaction mixture from 5 μg of total RNA, using a Superscript preamplification kit (Invitrogen) according to the manufacturer's instructions. The PCR conditions and the specific primers used for the amplification are as follows:

Amplification of sheep and cattle skeletal muscle mighty cDNA and bovine heart mighty cDNA was performed using the primers:

| | |
|---|---|
| Forward primer | |
| 5′ CACCATGGCGTGCGGGGCGACACTG 3′ | (SEQ ID No. 6) |
| Reverse primer | |
| 5′ GGATACATAGCTTGTTGGCCT 3′ | (SEQ ID No. 7) |

The PCR was carried out in the presence of Q solution (Qiagen) with initial denaturation at 94° C. for 1 min. Subsequently 35 cycles were performed of the following steps, 94° C. for 15 s, 60° C. for 45 s, 72° C. for 1 min, and 1 cycle of final extension at 72° C. for 5 min. PCR amplification of a mighty fragment from Belgian Blue and normal muscled cattle (FIG. 1).

Primers used:

| | |
|---|---|
| bcoo3291 Fwd | |
| 5′TGAAGCGGCCCATGGAGTTC 3′ | (SEQ ID No. 8) |
| bcoo3291 Rev2 | |
| 5′GGTGGGCTGGTCCTTCTTCATC 3′ | (SEQ ID No. 9) |

The PCR was performed in the presence of Q solution (Qiagen) and Taq polymerase with initial denaturation at 94° C. for 1 s followed by 35 cycles of 94° C. for 15 s, 62° C. for 30 s, 72° C. for 45 s and 1 cycle of final extension at 72° C. for 5 min.

The PCR products were run on a 1% agarose gel, stained with ethidium bromide and visualized. The results in FIG. 1 show that mighty is over expressed in the double muscled cattle compared to normal muscled animals. This result shows that mighty has a role in promoting muscle growth. Part A, of FIG. 2, shows that mighty gene expression is also up regulated in the heart tissue of the double muscle animals indicating that mighty is also able to regulate cardiac muscle growth and development as well as skeletal muscle growth and development. Part B of FIG. 2 shows the presence of mighty expression in ovine skeletal muscle.

Purification of the PCR products: The PCR reactions were run on 0.8% low melting point agarose gel and the gel containing the desired band was cut out. The DNA from the gel was purified using the Wizard PCR preps DNA purification system (Promega).

### EXAMPLE 2

### Cloning of Mighty cDNA

The purified cDNA was ligated in to pGEM-T easy vector according to the manufacturer's protocol (Promega). The ligation reaction was transformed into competent E. coli DH 5 alpha bacteria (Invitrogen) according to the manufacturer's protocol. The transformed bacteria were plated on Lennox L broth (LB) agar plates containing ampicillin (50 mg/litre), IPTG and X-gal. The white colonies were seeded in LB plus ampicillin media and the cultures grown overnight. The plasmid DNA was purified from the cultures using Qiagen mini plasmid kit (Qiagen). The plasmid DNA was digested with the restriction enzyme EcoRI, and analysed on an agarose gel. The positive clones were identified by the presence of the right size fragments. The positive clones were sent for sequencing for further confirmation. The ovine mighty polynucleotide sequence is provided in SEQ ID No. 1, and the corresponding polypeptide sequence is provided in SEQ ID No. 2. The bovine mighty polynucleotide sequence is provided in SEQ ID No. 3, and the corresponding polypeptide sequence as SEQ ID No. 4.

### EXAMPLE 3

### Generation of Murine Mighty Stable Cell Lines

The ORF of murine mighty was PCR amplified with the following primers:

| | |
|---|---|
| Fwd | |
| 5′ CACCATGGCGTGCGGGGCGACACTG 3′ | (SEQ ID No. 6) |
| Rev | |
| 5′ GGATACATAGCTTGTTGGCCT 3′ | (SEQ ID No. 7) |

The Pwo polymerase (Roche), Q solution (Qiagen), and mouse EST clone (Resgene) as the template, were used for the PCR reaction according to the manufacturer's recommendations. The PCR conditions were as follows: 35 cycles of 94° C. for 20 s, 60° C. for 30 s, 72° C. for 1 min and one cycle of 72° C. for 5 min.

The cDNA of the mouse mighty gene was purified through the Wizard PCR preparations

DNA purification system (Promega) and was cloned into the TOPO site of the pcDNA3.1 D/V5HisTOPO vector (Invitrogen) as per manufacturer's protocol. The recombinants were analysed by restriction digestion and the positive recombinant was sequenced.

For the stable transfection of C2C12 myoblasts with the mouse mighty construct, C2C12 myoblasts (1×10⁷) were washed twice in ice cold 1×HBS (140 mM NaCl, 0.77 mM Na₂HPO₄, 25 mM Hepes (7.1)) and resuspended in 0.5 ml ice cold 1×HBS and transferred to a precooled cuvette gap 0.4 cm (BioRad). 10 μg of linearised plasmid DNA was added (linearised with Sca I). After 5 minutes on ice, cells were mixed by agitation and the cuvette was pulsed at 0.24 kV at 960 μF capacitance with resistance set at 200Ω, and the time constant was an average of 36 ms. Cells were incubated for 10 minutes on ice and transferred to 10 ml of DMEM 10% FBS on a 10 cm dish and triturated up and down to break up cellular debris. Cells were then selected with geneticin (600 μg/ml) and individual clones selected. Clones expressing the transgene were identified by Western blot for the V5 tag in the plasmid.

### EXAMPLE 4

### Myoblast Proliferation Assay

Prior to assay C2C12 cells (Yaffe and Saxel) and transfected C2C12 clones were grown in DMEM media (Life Technologies, Grand Island, N.Y. USA), buffered with NaHCO₃ (41.9 mmol/l, Sigma Cell Culture Ltd, St Louis, Mo., USA) and gaseous CO₂. Phenol red (7.22 nmol/l, Sigma) was used as a pH indicator. Penicillin (1×10⁵ IU/l) and Streptomycin (100 mg/l, Sigma) were routinely added to media, as was 10% foetal bovine serum (Life Technologies Ltd).

Cell proliferation assays were conducted in uncoated 96-well Nunc microtitre plates. C2C12 cultures were seeded at 3×10³ cells/cm² in proliferation media. After a 24 hour attachment period media was decanted and fresh proliferation media added back to the plates.

Plates were then incubated in an atmosphere of 37° C. and 5% CO₂. A test plate was fixed at 0, 24, 48 and 72 hours post media change, and assayed for proliferation by the method of Oliver et al. (1989). Briefly, growth media was decanted and cells washed once with PBS then fixed for 30 min in 10% formol saline. The fixed cells were then stained for 30 min with 10 g/1 methylene blue in 0.01 M borate buffer (pH 8.5). Excess stain was removed by four sequential washes in borate buffer. Methylene blue was then eluted off the fixed cells by the addition of 100 ml of 1:1 (v/v) ethanol and 0.1 M HCl. The plates were then gently shaken and absorbance at 655 nm measured for each well by a microplate photometer (BioRad model 3550 microplate reader, BioRad, Hercules, Calif., USA).

The results in FIG. 4 show that the cells transfected with the mighty gene had a higher absorbance indicating a faster rate of growth compared to normal C2C12 cells. This result shows that mighty acts to up regulate the growth of myoblast cells.

### EXAMPLE 5

### Mighty Induced Hypertrophy

To assess the function of mighty in promoting myogenesis, the myoblasts stably expressing mighty were allowed to differentiate in low serum media. Immunostaining of MHC was performed to assess the morphology of the myotubes.

Mighty over-expressing cells and the parent cell line; C2C12, differentiated for 72 hours in DMEM 2% horse serum, were washed once in PBS then fixed with 70% ethanol:formaldehyde:glacial acetic acid (20:2:1) for 30 seconds, and then rinsed three times with PBS. Cells were then blocked overnight at 4° C. in TBS containing 1% normal sheep serum (NSS). Cells were incubated with the primary antibody, 1:100 dilution anti MHC, in TBS/1% NSS for 1 hour. Cells were washed (3×5 min) with TBST and incubated with the secondary antibody, 1:100 dilution sheep anti-mouse IgG in TBS/1% NSS for 30 minutes. Cells were washed as before and incubated with the tertiary antibody, 1:100 dilution of streptavidin-biotin peroxidase complex (RPN1051, Amersham), in TBS/1% NSS for 30 minutes. Cells were then washed again as before. MHC immunostaining was visualised using 3,3-diaminobenzidine tetrahydrochloride (DAB; Invitrogen) enhanced with 0.0375% CoCl₂ and then counterstained with Gills haematoxylin, mounted and photographed.

As shown in FIG. 5, expression of mighty confers an increase in the myonuclei number thereby indicating that mighty also promotes hypertrophy in muscle cells.

### EXAMPLE 6

### Analysis of Mighty Induced Hypertrophy

Methods:

Cell Culture

C2C12 cells or two Mighty overexpressing C2C12 clones; clone 7 and clone 11, were plated on permanox cover slips (Nunc) in DMEM 10% FBS (Invitrogen) at 15,000 cells/cm² for analysis of actively growing cells and 25,000 cells/cm² for differentiation studies.

After a 24 hour attachment period in an atmosphere of 5% CO2/37° C., media was changed either to growth media (DMEM/10% FBS) or to DMEM/2% HS (differentiation media) (Invitrogen) and the cells were allowed to differentiate for 60 and 72 hours. In order to visualise cells, cultures were fixed at the appropriate time points with 20 parts of 70% Ethanol/2 parts 40% Formaldehyde/1 part glacial acetic acid and then stained for 5 minutes with Gills haematoxylin (1:1) followed by one minute staining with 1% Eosin. Cover slips were then dehydrated in 100% Ethanol, cleared and permanently mounted using DPX on glass slides.

Human Myoblast Culture

Human skeletal muscle myoblasts were obtained from Clonetics, Cambrex N.J., USA. Cells were routinely grown in SkGM-2 media containing rhEGF, Dexamethasone, FBS, Glutimine and GA-1000 as per the manufacture's specifications.

For transfections and conditioned media experiments cells were plated at a density of 30,000 cells/cm². After a 24 hour attachment period cultures were either transfected with mighty-pcDNA3 and control vector or received conditioned media. Conditioned media consisted of DMEM/2% HS that had been subjected to 48 hours conditioning by the mighty clone 11 or control cells.

Twenty-four hours after transfection media was changed to DMEM/2% HS. Cultures were fixed with 20:2:1 fixative at 12, 24, 36, 48 hours.

Quantification of Hypertrophy in C2C12 Clones

The images were captured on a SPOT RT camera that was mounted on an Olympus microscope, using SPOT RT software v 3.5 designed for Windows and MAC. For the actively growing cells, the areas of 40 randomly selected cells per cell line were measured at 40× magnification. For the myotubes, measurements of the area, width and length of 40 randomly selected myotubes per cell line were taken, for 3 and 4 nuclei respectively also at 40× magnification. Data is expressed as mean +/− Standard Deviation.

FACS Analysis

Mighty overexpressing C2C12 clones 7 and 11, and the lacZ control C2C12 myoblasts were cultured in 10 cm dishes in DMEM 10% FBS. Cells were harvested using trypsin followed by centrifugation and fixed in 800 μl 70% ethanol/PBS. Fixed cells were then resuspended in 50 μl PBS+500 μl DNA extraction buffer (200 mM Na₂HPO₄; 100 mM citric acid) for 10 minutes at room temperature. DNA extraction buffer was replaced with DNA staining buffer (50 μg/ml propidium iodide; 50 μg/ml DNase-free RNase A in PBS), vortexed briefly to resuspend cells and incubated in the dark at room temperature for 30 minutes. Cells were then examined for propidium iodide fluorescence using a Becton-Dickinson FACScan flow cytometer (Becton-Dickinson) and forward angle light scatter, a measurement of cell size, and DNA content, for cell cycle analysis, was analysed using CellFit software (Becton-Dickinson).

Western Blotting

Mighty overexpressing C2C12 clones 7 and 11, and the lacZ expressing C2C12 myoblasts were cultured in 10 cm dishes in DMEM 10% FBS for 24 hours before being switched to differentiation media (DMEM 2% HS) for 0, 24, 48, 72 and 96 hours. Cells were harvested by trypsinisation and resuspended in 300 μl of lysis buffer (50 mM Tris (pH 7.5); 250 mM NaCl; 5 mM EDTA; 0.1% NP-40; 1× Protease inhibitor (Complete; Roche)). Cell extracts were passed through a 0.5 mm syringe needle ten times, centrifuged (14,000 g for 10 minutes) to pellet cell debris and the supernatant was used for western blotting. Protein concentration was determined using the Bio-Rad protein assay reagent (Bio-Rad) and 15 μg of protein was electrophoresed on a precast NuPAGE 4-12% Bis-Tris gel (Invitrogen) at 45 mA. The protein gel was then transferred to Trans-blot transfer membrane (Bio-Rad) using the Mini-Proteanil transfer system (Bio-Rad) at 50-60V for 2 hours. Membranes were then blocked overnight in 5% milk in TBST. Primary antibodies were diluted as follows in 5% milk in TBST: p21, 1:400 dilution of purified mouse monoclonal anti-p21 antibody (SX118; PharMingen); MyoD, 1:200 dilution of purified rabbit polyclonal anti-MyoD antibody (sc-304; Santa Cruz); MHC, 1:2000 dilution of purified mouse monoclonal anti-MHC antibody (MF-20; gift from Dr Donald Fischman); α-tubulin, 1:4000 dilution of purified mouse monoclonal anti-α-tubulin antibody (DM1A; Sigma); and incubated at room temperature for 3 hours. Membranes were washed 5× for 5 min in TBST and incubated for a further 1 hour at room temperature in 5% milk in TBST containing anti-mouse IgG HRP conjugate (P0447;Dako) or anti-rabbit IgG HRP conjugate (P0448; Dako) at 1:2000 dilution. Membranes were then washed 5× for 5 minutes in TBST and HRP activity was detected using the Western Lightning Chemiluminescence Reagent Plus (Perkin Elmer).

Results:

Mighty Overexpressing Myoblasts and Myotubes Show Hypertrophy Over Control Cells.

Actively growing mighty overexpressing clones appear hypertrophied when compared to control cells. Using quantitative image analysis actively growing mighty overexpressing clones have a significantly larger area than control cells (FIG. 6A). To confirm this result, myoblast hypertrophy, or relative cell size, was measured by flow cytometry using forward angle light scatter. This analysis demonstrated an increase in cell size in clone 11 and clone 7 respectively over control cells (FIG. 6B).

Differentiated mighty overexpressing clones also appear hypertrophied as compared to control cells. Given that an explanation for hypertrophy may be an increase in the number of cells fused per myotube, analysis was performed to compare the size of myotubes with the same number of nuclei between the mighty overexpressing clones and control cells. Using quantitative image analysis, myotube hypertrophy was evident in mighty overexpressing clones. Myotube area, width and length of tri and tetra nucleated myotubes were compared. Mighty overexpressing clones demonstrated an increase in area, width, and length over control cells in both tri and tetra nucleated myotubes (FIG. 6C-6E).

Mighty Overexpressing Clones Differentiate Earlier than Control Cells

The differentiation phenotype of mighty overexpressing clones was determined using immunocytochemistry for MHC to visualise myotube formation. Upon switching mighty overexpressing clones to differentiation media multinucleated myotubes are evident in mighty overexpressing clones by 60 hours while multinucleated myotubes do not become evident in control cultures until 72 hours. By 72 hours mighty overexpressing appear almost totally differentiated while control cells appear to be forming only nascent myotubes (FIG. 7A). These results were confirmed using western blotting for differentiation markers. Early, mid and late differentiation markers p21, myoD and MHC respectively were used to investigate myogenic differentiation gene expression (FIG. 7B). p21 expression was increased at all time points, indicating that p21 expression occurs earlier and to a greater extent in mighty overexpressing clones. MyoD expression increases earlier in mighty overexpressing clones with myoD expression increased over control cells at 0, 24 and 48 hours of differentiation. MyoD levels were equivalently high by 72 hours in all cell lines. MHC expression occurs by 24 hours in clone 11 and is expressed to a higher level in both clones at 48 hours. This expression is equivalent by 72 hours in all cell lines. The earlier and increased expression of myogenic differentiation markers is concurrent with the histochemical results. These results indicate that overexpression of mighty results in an enhanced differentiation phenotype.

### EXAMPLE 7

### Mighty in Muscle Development

Methods:

Isolation of Satellite Cells (Quiescent)

Satellite cells (SC) were isolated from the hind limb muscles of 4 week old wild type mice be Percoll density centrifugation. Muscles were minced, and digested in 0.2% collagenase type 1A for 90 min. Cells were released from below the basal lamina by gentle trituration and then filtered (70 μm). The cell suspension was then overlaid onto 70%/40% Percoll gradients and centrifuged at 1,600 rpm for 20 min. The interface between the 70% and 40% Percoll solutions containing satellite cells was recovered and the cells washed in PBS. To extract protein for Western blotting the cells were resuspended in lysis buffer and passed through a 0.45 mm gauge needle to lyse the cells. Cellular debris was removed by centrifugation at 10,000 rpm for 10 min, and the resulting protein solution stored at −80° C.

Isolation of Primary Myoblasts (Activated Satellite Cells)

Hind limb muscles were removed from 4-week old mice, minced thoroughly and digested with 0.2% collagenase 1A in DMEM (no serum) at 37° C. with shaking (70 rpm) for 90 min. The digest was triturated with a 10 ml pipette repeatedly until no lumps were visible. The suspension was then filtered through a 100 μm and then a 70 μm filter. The filtered suspension was then centrifuged at 4,000 rpm for 10 min and the pellet resuspended in 8 ml of warm proliferation media [DMEM, 20% foetal calf serum (FCS), 10% horse serum (HS); 1% chick embryo extract (CEE)]. The cell suspension was pre-plated on uncoated 10 cm plates for 1.5 h, then transferred to 10% matrigel plates and incubated for 48 h at 37° C. After 48 h the media was changed to either DMEM+10% FCS. Cells were collected after 24 h in actively growing conditions. To extract protein for Western blotting the cells were suspended in lysis buffer and passed through a 0.45 mm gauge needle to lyse the cells. Cellular debris was removed by centrifugation at 10,000 rpm for 10 min, and the resulting protein solution stored at −80° C.

Western Analysis for Mighty

Pre-cast polyacrylamide gels (Invitrogen, NuPage 4-12% Bis-Tris) were used for protein separation. 15 μg of protein was separated by SDS-PAGE (4-12%) and transferred to a nitrocellulose membrane by electroblotting. Protein present on the nitrocellulose membrane was detected using Ponceau S stain to ensure even loading had occurred. The membrane was incubated in 0.3% BSA/1% PVP/1% PEG/TBS-T for 3 h at RT to block non-specific antibody binding. The membrane was then incubated with rabbit anti-mighty antibody 1:5000 dilution in 0.3% BSA/1% PVP/1% PEG/TBS-T at 4° C. overnight, with gentle shaking. Between antibody incubations the membrane was washed 5×5 min each in TBS-T. The nitrocellulose membrane was then incubated with goat anti-rabbit conjugated to Horseradish Peroxidase (HRP) (Amersham) 1:2000 dilution in 0.3% BSA/1% PVP/1% PEG/TBS-T for 1 h. HRP activity was detected with ECL reagent (Western Lightning Chemiluminescense Reagent Plus).

Results:

Expression of Mighty in Murine Satellite Cells

To determine the pattern of mighty protein expression, total protein was extracted from quiescent satellite cells, actively growing myoblasts. Mighty protein expression levels were analysed by Western blotting (FIG. 8). In quiescent satellite cells, mighty protein expression was not detected. However, mighty protein was detected in actively growing myoblasts (activated satellite cells).

### EXAMPLE 8

### Mighty in Muscle Regeneration

Methods:

Notexin Induced Regeneration

Six week old wild type mice were anaesthetised by intraperitoneal injection with ketamine/rompun (Ketamine Hydrochloride 100 mg/ml, Xylazine Hydrochloride 20 mg/ml at 0.1 ml/6 gm body weight). The fur was trimmed from the area over the right tibialis anterior (TA) muscle and a small incision made over the muscle. Using a 100 μl syringe (Hamilton Co.) 0.1 μg of notexin (Notechis scutatus scutatus) (Venom Supplies Pty. Ltd., Tanunda, South Aust) in 10 μl was injected into the right TA. The incision was closed with Michelle clips. Mice were euthanised on respective days after the injury by CO₂ asphyxiation followed by cervical dislocation. Right and left TA muscles were carefully dissected out, weighed and processed for immunohistochemistry analysis.

Immunohistochemistry for Mighty

Mouse TA muscle and sheep heart tissue was isolated, embedded in OCT and frozen in isopentane chilled in liquid nitrogen. Cryosections were cut at 10 μm and the slides frozen at −20° C. until used. The sections were permeabilised in PBS, 0.1% Triton X-100 for 30 min at room temperature and incubated with primary anti-mighty antibody at 1:100 dilution in PBS, 10% normal donkey serum, 1% BSA, 0.1% Triton X-100 overnight at 4° C. After washing 3×4 min in PBS, the slides were incubated in PBS, 5% normal donkey serum, 1% BSA, 0.1% Triton X-100 for 1 h to reduce non specific binding of the secondary antibody. The sections were incubated with biotinylated anti-rabbit secondary antibody (Amersham, UK) at 1:300 dilution for 1 h at room temperature. Following washes in PBS, the sections were finally incubated in streptavidin-conjugated Alexa fluor 488-labeled tertiary antibody (Molecular Probes, USA) for 1 h at room temperature, washed in PBS, counter stained with DAPI (4′,6-diamidino-2-phenylindole, dihydrochloride, Molecular Probes, USA), mounted in DAKO fluorescent mounting medium and analysed for mighty expression. Control sections were incubated with either no primary antibody or rabbit control IgG and then processed as described above.

Results:

Expression of Mighty During Murine TA Muscle Regeneration

To investigate the expression of mighty during muscle regeneration in wild type mice, injury was induced by the injection of Notexin into the right tibialis anterior (TA) muscle. On various days post-injection, mice were euthanised and TA muscles collected. To determine the pattern of mighty expression immunohistochemistry was performed (FIG. 9). On day one following injury there was extensive damage to the integrity of the muscle fibres, with no change in the level of mighty expression observed. By day 5 mighty expression level had substantially increased compared to day 0, with the levels peaking on day seven post-injury. The level of mighty expression by day 28 was comparable to control, non-injured muscle.

### EXAMPLE 9

### Expression of Mighty in Postinfarction Heart Tissue in Sheep

Methods:

Myocardial infarction in sheep heart was induced by the published method described in Sharma et al, 1999. Heart tissue was collected on day 0 (non-infarcted), and on days 2, and 6, post-infarction. To determine the pattern of mighty expression immunohistochemistry was performed on the heart tissue sections as described in the muscle regeneration example (example 8).

Results:

In non-infarcted heart tissue the expression of mighty was homogeneous and restricted to the cells located within interstitium surrounding the cardiomyocytes. Mighty did not appear to be expressed by the cardiomyocytes. Two days post-infarction, expression of mighty was similar to that in control heart. By day 6 following infarction there was a substantial increase in the number of infiltrating cells primarily within the infarcted zone. The expression of mighty was restricted to the infiltrating cells located closest to areas of necrotic myocardium. Where infiltrating cells adjoined surviving myocardium, expression levels remained similar to control. Infiltrating cells distal to the infarcted area did not express mighty (FIG. 10).

The presence of mighty in interstitial fibroblasts and infiltrating cells in the region of necrotic myocardium confirms that Mighty is involved in the process of healing.

### EXAMPLE 10

### Effect of Mighty on Differentiation and Hypertrophy of Human Myoblasts

Results:

Increased Differentiation in Human Myoblasts Transfected with Mighty

To determine the effect of mighty over-expression on the differentiation in human myoblasts, human myoblasts were transfected with mighty-pcDNA3 or pcDNA3 alone (control) and cultured in differentiation media for 12 h. To determine the number of differentiating myotubes immunocytochemistry using myosin heavy chain (MHC) specific antibodies was performed. After 12 h in differentiating conditions the number of MHC positive myotubes was greater in the mighty transfected human myoblasts compared to control myoblasts (FIG. 11).

Hypertrophy of Human Myoblasts Transfected with Mighty

Human myoblasts were transfected with Mighty-pcDNA3 or pcDNA3 alone (control) and cultured under differentiating conditions for 12 h. To determine hypertrophy, immunocytochemistry using myosin heavy chain (MHC) specific antibodies was performed and the number of myonuclei per MHC positive myotube was counted (FIG. 12A). Results show that fewer mighty transfected human myotubes contain only 1-3 myonuclei with a greater number of myotubes containing 4-9 myonuclei as compared to the control. Hypertrophy of MHC expressing myotubes was also assessed by measuring the widths of myotubes containing 5-9 myonuclei (FIG. 12B). The average width of mighty transfected myotubes was greater compared to control myotubes (FIG. 12B).

Hypertrophy of Human Myoblasts Treated with Conditioned Media from Mighty Over-Expressing C2C12 Cells

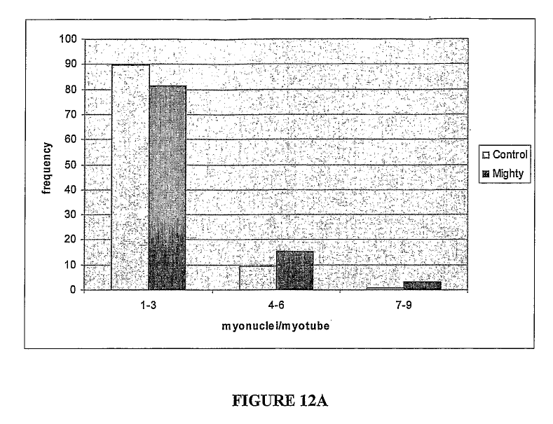

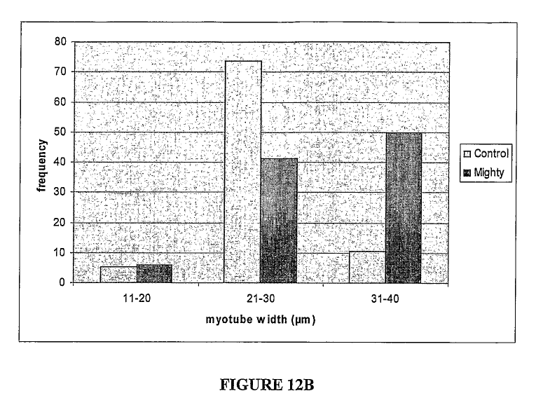

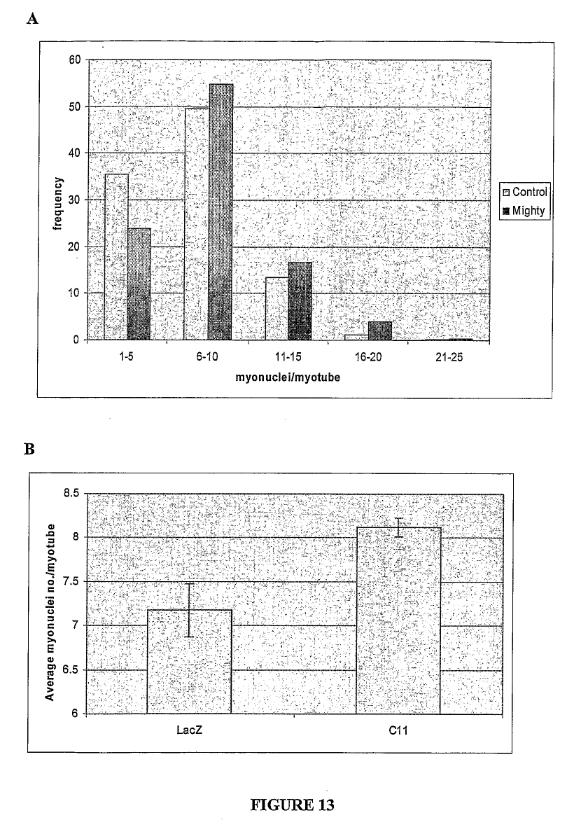

To demonstrate the phenomenon of accelerated differentiation and hypertrophy induced by mighty, human myoblasts were cultured under differentiating conditions with conditioned media from Mighty over-expressing C2C12 cells and LacZ (control) C2C12 cells for 48 h. The level of hypertrophy was assessed as above. Human myoblasts cultured with conditioned media from mighty over-expressing C2C12 cells contained fewer myotubes with only 1-5 myonuclei and greater number of myotubes with 16-25 myonuclei, compared to control treated myoblasts (FIG. 13A). The average number of myonuclei was greater in human myoblasts treated with conditioned media from mighty over-expressing C2C12 cells compared to the control (FIG. 13B).

Hypertrophy of MHC expressing myotubes was again also assessed by measuring the widths of myotubes. MHC expressing myotubes containing 8 myonuclei were measured (FIG. 13). The average width of myotubes treated with conditioned media from mighty over-expressing C2C12 cells was greater compared to control myotubes

Increased Differentiation in Human Rhabdomyosarcoma (Rd) Cells Treated with Conditioned Media from Mighty

Methods:

Human RD (Rhabdomyosarcoma) cells were obtained from the ATCC (Rockville, Md.). RD cells were grown prior to assay in Dulbecco's Modified Eagle Medium (DMEM; Invitrogen), buffered with 41.9 mM NaHCO₃ (Sigma Cell Culture Ltd) and 5% gaseous CO₂. 7.22 nM Phenol red (Sigma) was used as a pH indicator. 1×10⁵ IU/L penicillin (Sigma), 100 mg/L streptomycin (Sigma) and 10% Fetal Bovine Serum (FBS; Invitrogen) were added to media. RD cells were plated on permanox chamber slides at a density of 30,000 cells/cm. 24 hours later they were treated with conditioned media (mighty clone 11) as mentioned for human myoblasts and the cells fixed after 72 hours. Immunocytochemistry for MHC was performed.

Result:

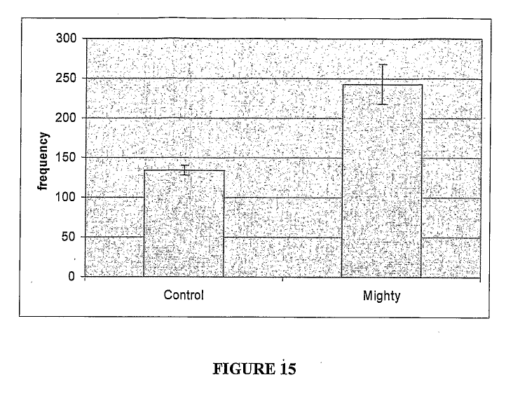

Human RD cells were treated with mighty conditioned media and control media and allowed to differentiate for 72 hours. The cells were immunostained for MHC to assess the extent of differentiation. The number MHC positive myotubes was higher in the treated cells as compared to the control cells indicating that conditioned media from mighty overexpressing clones can accelerate differentiation (FIG. 15).

### EXAMPLE 11

### Cloning of the Murine Mighty Promoter

Methods:

The 2.1 kb of 5′ upstream sequence was amplified using the mouse genomic DNA and the following primers:

Rev

(SEQ ID No. 10)

5′ AGA TCT GAT CCA ACT CTT CAG CTA C 3′
Fwd

(SEQ ID No. 11)

5′ GCT AGC CCA CAT TCA CTG TGC AAG 3′

The PCR was carried out using Q solution (Qiagen) and Expand long DNA polymerase (Roche) according to the manufacturer's protocol. The PCR conditions were, 35 cycles of 95° C. for 15 s, 52° C. for 30 s, 68° C. for 3 min and one cycle of final extension at 68° C. for 7 min.

The PCR product was analysed on a 0.8% agarose gel and purified through a Wizard purification column (Promega). The purified DNA fragment was cloned into the pGEM-T easy vector as mentioned above. The positive recombinants were selected and analysed by restriction digestion and sequencing. The 2.1 kb fragment was cut out from the pGEM-T easy vector by BglII and NheI enzymes for cloning into a luciferase reporter vector pGL3B. The pGL3B was digested with BglII and NheI and the 2.1 kb mighty promoter fragment was ligated to it using T4 ligase. The E. coli DH 5 alpha was transformed with the ligation reaction and plated on LB agar plus ampicillin plates. The cultures were grown in LB plus ampicillin media and plasmid DNA purified as mentioned previously. The plasmid DNA was analysed by restriction digestion and the positive recombinants identified. The positive recombinant (2.1 construct) was confirmed by sequencing (SEQ ID No. 5). For transfection experiments, the DNA was purified using Qiagen Maxi Prep Kit (Qiagen).

The mighty promoter sequence is shown in SEQ ID No. 5. The mighty promoter sequence was also analysed for known transcription factor binding sites (FIG. 3). These sites show crucial parts of the promoter sequence.

### EXAMPLE 12

### Example of the Mighty Promoter Activity in Various Cell Lines Including Human

Methods:

1 kb of the mighty upstream sequences (a fragment of 2.1 kb mighty promoter) was cloned into the luciferase reporter vector pGL3-basic (Promega). The 1 kb mighty promoter fragment was derived from the 2.1 kb promoter sequence by restriction digestion. A Sca1, BglII digestion was performed on 2.1 mighty promoter construct to excise the ˜1 kb fragment. This fragment was then cloned into the SmaI and BglII sites within the multiple cloning sites of pGL3b in the correct orientation to drive luciferase expression.

Transfections were performed with 2 μg of this promoter construct and 0.5 or 1 μg of the β-galactosidase (β-gal) expression plasmid pCH110 (Amersham) for normalisation of transfection efficiency. C2C12 myoblasts, NIH3T3, CHO, RD (human Rhabdomyosarcoma cell line) and primary ovine myoblasts were transfected with the vectors above using LipofectAMINE 2000 reagent (LF2000, Invitrogen) according to the manufacturer's protocol. Briefly cell lines were plated on a 6-well cell culture dishes (Nunc) at 15,000 cells/cm² in appropriate growth media and incubated overnight at 37° C., in 5% CO₂ before transfection. Constructs were diluted in 250 μl of DMEM without serum per well. LF2000 was diluted at 5-8 ul in 250 μl of DMEM without serum per well. Diluted DNA and LF2000 was then mixed and incubated at room temperature for 20 minutes. The DNA/LF2000 mix was then added to wells containing cells in 2 ml of appropriate growth media. Cell lines were incubated with the transfection mix at 37° C., in 5% CO₂ overnight, after which media was replaced with either growth or differentiation media. Cells were then rinsed twice in 5 ml of PBS per well and lysed in 300-500 μl of Reporter lysis buffer (Promega). Ten μl of cell lysate was used to detect luciferase activity using the luciferase assay system (Promega) as per the manufacturer's protocol. Fifty μl of cell lysate was used to perform β-gal assays using the β-galactosidase enzyme assay system with reporter lysis buffer (Promega) as per the manufacturer's protocol. The luciferase values were normalised to β-gal values to normalise for transfection efficiency.

Results:

1 kb of the murine mighty upstream sequence was transfected into a variety of cell lines. These include C2C12 myoblasts, primary ovine myoblasts, NIH3T3 fibroblasts, and Chinese Hamster Ovary (CHO) cells (FIG. 16A) and human RD cells (FIG. 16B). The murine mighty promoter showed strong activity in all of these cell lines. Therefore the mighty promoter shows strong expression in cell lines derived from different tissue types and species including human.

### EXAMPLE 13

### Dose Dependent Inhibition of the Mighty Promoter by Myostatin

Methods:

Method of transfection of mighty promoter in C2C12 myoblast was as described above except for the treatment of the transfected cells with myostatin protein. Twenty four hours after the transfection, the cells were treated with 4 and 8 μg/ml of recombinant myostatin in growth media. The cells were harvested after 24 h of the treatment. The luciferase and β-galactosidase activity were determined as described above.

Results:

The 1 kb murine mighty promoter was transfected into C2C12 myoblasts and treated with increasing concentrations of 4 and 8 μg/ml of recombinant myostatin protein. Activity of the 1 kb mighty promoter is inhibited in the presence of 4 and 8 μg/ml myostatin protein (39.23+/−0.99% and 58.22+/−1.00% respectively). Moreover increasing concentrations of myostatin inhibited the mighty promoter to increasing extents (FIG. 17). Therefore myostatin inhibits the mighty promoter in a dose dependent manner.

### EXAMPLE 14

### Myostatin Mimetic (335) Rescues the Effect of Myostatin on Mighty Promoter

Transfection of Ovine Satellite Cells

Ovine satellite cells were grown in DMEM+10% FBS as described above. The cells were transfected in a 24 well plate with 0.4 μg of the 1 kb mouse mighty promoter construct and 0.1 μg of pCH110 (SV40-galactosidase control vector, Amersham) using Lipofectamine 2000 according to the manufacturer's instruction (Invitrogen). 24 hours later the media was changed to DMEM+10% FBS+3 μg/ml wild type recombinant myostatin or 335 (15 mg) or myostatin and 335. 24 hours after the media change the cell extracts were made and luciferase assays (Promega) were performed according to the established protocols). The assays for beta-galactosidase activity were done according to the protocol (Promega). Luciferase activity was normalised to β-galactosidase activity.

Result

Ovine myoblasts were transfected with 1 kb mighty promoter and β-galactosidase vector and treated with myostatin or myostatin mimetic or both. As shown in FIG. 18, upon treatment with wild type myostatin a 33% inhibition of mighty promoter activity was seen. When the cells were treated with both myostatin and 5 molar excess of 335 a dominant negative mimetic for myostatin only 19% inhibition of mighty promoter activity was observed. Thus the dominant negative myostatin mimetic 335 can rescue the myostatin mediated inhibition of mighty promoter.

### EXAMPLE 15

### Truncation Analysis of the Mighty Promoter

Methods:

The Mighty 0.6 kb promoter was amplified using the forward primer with a NheI restriction site5′-GCTAGCGTGATCCGATTAATGGCC-3′ and the reverse primer with a BglII restriction site 5′-AGATCTGATCCAACTCTTCAGCTAG-3′. The Mighty 0.4 kb promoter was amplified using the forward primer with a NheI restriction site 5′-GCTAGCCCCTTTAGAATCACCTC-3′ and the reverse primer with a BglII restriction site 5′-AGATCTGATCCAACTCTTCAGCTAG-3′. The Mighty 0.315 kb promoter was amplified using the forward primer with a NheI restriction site5′-GCTAGCCGCAGGTGCGAAAGACCTC-3′ and the reverse primer with a BglII restriction site 5′-AGATCTGATCCAACTCTTCAGCTAG-3′. The Mighty 0.287 kb promoter was amplified using the forward primer with a NheI restriction site5′-GCTAGCTCCGGCAGAGAGCGTGAAG-3′ and the reverse primer with a BglII restriction site 5′-AGATCTGATCCAACTCTTCAGCTAG-3′. The Mighty 0.209 kb promoter was amplified using the forward primer with a NheI restriction site 5′-GCTAGCAGACCGGCCTACTTCTTC-3′ and the reverse primer with a BglII restriction site 5′-AGATCTGATCCAACTCTTCAGCTAG-3′. These truncations were cloned into the NheI and BglII restriction sites of pGL3b in the correct orientation to drive luciferase expression.

Results

The mighty promoter fragments (from 0.2 kb to 2.1 kb) were transfected in to C2C12 myoblasts and luciferase activity assayed. The luciferase activity was normalised to β-galactosidase activity. The results as shown in FIGS. 19 (A and B), show that the promoter activity is maximal from 300 bp to 1 kb of the mighty promoter. There appears to be a slight decrease in promoter activity between 1 kb and 2.1 kb.

### EXAMPLE 16

### Mighty Antibody Production

Methods

Polyclonal antibody against full length bovine mighty protein was raised in rabbits. First, the bovine mighty cDNA was cloned into pRSET B vector (Invitrogen) and finally transformed into E. coli BL21 star (Invitrogen) according to the manufacturer's protocol. Expression of the recombinant protein was induced by adding 0.5 mM IPTG and continuing incubation for two and half hours. Bacteria were collected by centrifugation, re-suspended in 40 ml of lysis buffer (6M guanidine HCl, 20 mM Tris pH 8.1, 5 mM 2-mercaptoethanol), and then sonicated. The lysate was centrifuged at 10,000 g for 30 min and recombinant protein purified from the supernatant using a Ni-agarose affinity protocol (Qiagen, Valencia, Calif.). The fractions were pooled and dialysed against two changes of 50 mM Tris pH8.0 containing 200 mM NaCl and 5% glycerol for 90 min at 4° C. The purified mighty protein was emulsified with Freund's adjuvant and injected into each rabbit (341 □g/rabbit). Subsequently two booster doses containing 170 □g mighty protein/injection were given to each rabbit.

Blood from the inoculated rabbits was collected and centrifuged at 2000 rpm for 15 minutes at 4° C. Serum was separated from clot. 2.5 ml of Protein-A Agarose was used to pack a column to purify IgG fraction of antibodies. The column was washed with 25 ml of 100 mM Tris pH 8.0. The pH of serum was adjusted with 1/10th volume of 1.0M Tris (pH 8.0) and 5.5 ml of the serum was passed through the column. The recovered fraction was passed through the column again. Next, the column was washed with 25 ml of 100 mM Tris (pH 8.0). A second wash was performed using 25 ml of 10 mM Tris (pH 8.0). The antibodies were eluted using 100 mM glycine (pH 3.0). The eluate was collected in tubes containing 50 μl of 1M Tris (pH 8.0) and mixed gently. Immunoglobulin-containing fractions were identified by using Bradford method for protein estimation.

Peptide Specific Mighty Antibody

Antibodies against an 18 mer mighty peptide (173-190 AA) were raised by QED Bioscience, Inc., CA, USA on our specifications.

Western Blotting

Western blot analysis was carried out to verify the antibodies raised against full length bovine mighty protein and an 18mer mighty peptide (173-190). Specifically, protein extracts from the E. coli cells expressing recombinant bovine mighty protein or purified recombinant mighty protein were resolved on a 4-12% NuPAGE (Invitrogen) gel according to the manufacturer's instructions. The mighty protein antibodies were used at 1:10,000 for Western blotting whereas 1:5000 dilution was used for peptide antibodies.

Results

Both, peptide and mighty protein antibodies specifically recognized an expected size that is 35 kDa protein band on the Western blot confirming that these antibodies are specific for mighty protein (FIG. 20).

Wherein the foregoing description reference has been made to integers or components having known equivalents and such equivalents are herein incorporated as if individually set forth.

Although the invention has been described by way of example and with reference to possible embodiments thereof, it is to be appreciated that improvement and or modifications may be made thereto without departing from the scope thereof.

### REFERENCES

Eryl Liddell, J. and Cryer, A. (1996) A Practical Guide to Monoclonal Antibodies. Wiley.
Javois, Lorette C. (1999) Immunocytochemical Methods and Protocols. Humana Press.
Kambadur, R., Sharma, M., Smith, T. P. L. and Bass, J. J. (1997) Mutations in myostatin (GDF-8) in doubled muscled Belgian Blue and Piedmontese Cattle. Genome Res. 7. 910-916.
Michael P. Spiller, Ravi Kambadur, Ferenc Jeanplong, Mark Thomas, Julie K. Martyn, John J. Bass and Mridula Sharma (2002). Myostatin is a downstream target gene of basic helix loop helix transcription factor, MyoD. Mol & Cellular Biology 22: 7066-7082.
Oliver, M. H., Harrison, N. K., Bishop, J. E., Cole, P. J. and Laurent, G. J. (1989). A rapid and convenient assay for counting cells cultured in microwell plates: application for assessment of growth factors. Journal of Cell Science 92, 513-518.
Yaffe, D. and Saxel, O. (1977). Serial passaging and differentiation of myogenic cells isolated from dystrophic mouse muscle. Nature 270, 725-727.

1. An isolated polypeptide comprising a sequence selected from the group consisting of SEQ ID No. 2 and a sequence with 95% identity thereto.

2. An isolated polynucleotide that encodes an isolated polypeptide comprising a sequence selected from the group consisting of SEQ ID No. 2 and a sequence with 95% identity thereto.

3. An isolated polynucleotide according to claim 2, selected from the group consisting of SEQ ID No. 1, SEQ ID No. 3, the complement of SEQ ID NO: 1, the complement of SEQ ID NO: 3, the reverse complement of SEQ ID NO: 1, the reverse complement of SEQ ID NO: 3, the reverse sequence of SEQ ID NO: 1, and the reverse sequence of SEQ ID NO: 3.

4. (canceled)

5. An isolated polynucleotide comprising a nucleotide sequence that differs from SEQ ID No: 1 or SEQ ID No. 3 as a result of silent substitution(s) or substitution(s) that results in conservative substitution(s) in the resulting amino acid.

6. An isolated polypeptide encoded by a polynucleotide comprising a nucleotide sequence that differs from SEQ ID No: 1 or SEQ ID No. 3 as a result of silent substitution(s) or substitution(s) that results in conservative substitution(s) in the resulting amino acid.

7. A fusion protein comprising at least one polypeptide according to claim 1 or a fragment thereof and additional amino acids.

8. A vector comprising a polynucleotide according to any one of claims 2 or 5.

9. The vector according to claim 8, further comprising, in the 5′-3′ direction:
a) a gene promoter sequence; and
b) a gene termination sequence.

10. The vector according to claim 8, wherein the polynucleotide is in a sense orientation.

11. The vector according to claim 8, wherein the polynucleotide is in an antisense orientation.

12. A host cell containing a vector according to claim 8.

13. A composition for regulating muscle growth, comprising an active ingredient selected from the group consisting of:
a) a polynucleotide comprising SEQ ID No. 1, SEQ ID No.3, or SEQ ID NO: 5,
b) a fragment or variant of (a),
c) a polynucleotide having at least 95% sequence identity to (a),
d) a complement of any one of (a) to (c),
e) a reverse complement of any one of (a) to (c),
f) an antisense polynucleotide of any one of (a) to (c),
g) a polypeptide encoded by any one of (a) to (c),
h) a polypeptide comprising SEQ ID No. 2 or SEQ ID No. 4,
i) a fragment or variant of (g) or (h), and
j) a polypeptide having at least 95% sequence identity relating to (g) or (h); and
a pharmaceutically acceptable diluent, excipient or carrier.

14. (canceled)

15. A composition for modulating mighty gene expression comprising a compound capable of binding to a polynucleotide selected from the group consisting of:
a) SEQ ID No. 1, SEQ ID No. 3, or SEQ ID No. 5,
b) a polynucleotide that encodes a polypeptide of SEQ ID No. 2 or SEQ ID No. 4,
c) a polynucleotide having at least 95% sequence identity to (a) or (b),
d) a complement of any one of (a) to (c),
e) a reverse complement of any one of (a) to (c), and
f) a fragment or variant of any one of (a) to (e); and
a pharmaceutically acceptable diluent excipient or carrier.

16. The composition according to claim 15 wherein the compound is an anti-sense polynucleotide.

17. The composition according to claim 15 wherein the compound is an interfering RNA molecule.

18. The composition according to claim 17 wherein the interfering RNA molecule is an RNAi or siRNA molecule.

19. The composition according to claim 15, wherein the compound is myostatin.

20. The composition according to claim 15, wherein the compound is a myostatin mimetic.

21. The composition according to claim 20, wherein the myostatin mimetic is a myostatin peptide C-terminally truncated at or between amino acid positions 330 and 350.

22. The composition according to claim 20, wherein the myostatin mimetic is a myostatin peptide C-terminally truncated at a position selected from the group consisting of amino acid positions 330, 335, and 350.

23. The composition according to claim 15, wherein the compound is an antibody.

24. (canceled)

25. (canceled)

26. (canceled)

27. (canceled)

28. A method of regulating muscle growth of an organism, comprising administering to said organism a composition according to claim 13 or 15.

29. The method according to claim 28, for the production of increased muscle mass in said organism.

30. The method according to claim 28, for the treatment or prophylaxis of a disease associated with muscle growth in said organism.

31. The method according to claim 30, wherein the disease is associated with muscle atrophy.

32. The method according to claim 30, wherein the disease is selected from the group consisting of muscular dystrophy, muscle cachexia, atrophy, hypertrophy, muscle wasting associated cancer or HIV, amyotrophic lateral sclerosis (ALS), and diseases associated with cardiac muscle growth, including infarct.

33. A method according to claim 28, for promoting muscle regeneration after muscle injury in said organism.

34. (canceled)

35. (canceled)

36. (canceled)

37. (canceled)

38. (canceled)

39. A transgenic animal comprising a vector according to claim 8.

40. The transgenic animal according to claim 39, wherein said animal has an increased muscle mass.

41. The transgenic animal according to claim 39, selected from the group consisting of a sheep, cow, bull, deer, poultry, turkey, pig, horse, mouse, rat and human.

42. A method of predicting muscle mass in an animal, comprising the steps of:
obtaining a sample from the animal,
determining the gene expression level from a polynucleotide having a sequence of SEQ ID No. 1 or SEQ ID No. 3, a polynucleotide having at least 95% sequence identity to SEQ ID No. 1 or SEQ ID No. 3, or a fragment or variant thereof; or determining the amount of a polypeptide having a sequence of SEQ ID No. 2 or SEQ ID No. 4, a polypetide having at least 95% sequence identity to SEQ ID No. 2 or SEQ ID No. 4, or a fragment or variant thereof,
comparing the gene expression level or amount of polypeptide to an average; and
predicting the muscle mass of said animal based on the gene expression level.

43. The method according to claim 42, wherein the level of gene expression is determined using RTPCR or northern analysis.

44. The method according to claim 43, wherein the amount of the polypeptide is determined using ELISA or Western blot analysis.

45. A method of detecting a variant of mighty, comprising the use of a nucleotide sequence selected from the group consisting of:
a) SEQ ID No. 1, SEQ ID No. 3, or SEQ ID No. 5,
b) a polynucleotide that encodes a polypeptide of SEQ ID No. 2 or SEQ ID No. 4,
c) a polynucleotide having at least 95% sequence identity to (a) or (b),
d) a complement of any one of (a) to (c),
e) a reverse complement of any one of (a) to (c), and
f) a fragment or variant of any one of (a) to (e), to screen a sample from an organism for the variant of mighty.

46. The method according to claim 45, wherein the variant is a polymorphism.

47. The method according to claim 46, wherein the polymorphism is a single nucleotide polymorphism.

48. The method according to claim 45, wherein the variant of mighty is associated with an altered muscle phenotype.

49. A method of breeding an animal having improved muscle mass comprising the steps of:
selecting one or more animals predicted to have an increase in muscle mass using the method according to claim 42, and
breeding the one or more animals predicted to have an increased muscle mass to produce an animal having an improved muscle mass.

50. The method according to claim 49, wherein the animal is selected from the group consisting of a sheep, cow, bull, deer, poultry, turkey, pig, horse, mouse, rat, fish and human.

51. An antibody that preferentially binds a polypeptide having a sequence of SEQ ID NO. 2 or SEQ ID NO. 4 or a polypeptide having at least 95% sequence identity to SEQ ID NO. 2 or SEQ ID NO. 4.

52. An antigenic fragment of a polypeptide comprising a sequence of SEQ ID NO. 2 or SEQ ID NO. 4 in the production of an antibody that preferentially binds a sequence of SEQ ID NO. 2 or SEQ ID NO. 4 or a polypeptide having at least 95% sequence identity to SEQ ID NO. 2 or SEQ ID NO. 4.

53. An isolated polynucleotide selected from the group consisting of:
a) a polynucleotide comprising the sequence of SEQ ID No: 5,
b) a polynucleotide comprising at least 95% sequence identity to SEQ ID No. 5, and
c) a polynucleotide comprising a fragment or variant of (a) or (b) having promoter activity.

54. (canceled)

55. An isolated polynucleotide according to claim 53, comprising at least the 200 nucleotides upstream of the mighty initiation site.

56. An isolated polynucleotide according to claim 53, comprising a fragment selected from the group consisting of those 209, 287, 315, 400, 600, 1000 and 2100 nucleotides upstream of the mighty initiation site.

57. A vector comprising a polynucleotide according to claims 53.

58. An isolated host cell containing a vector according to claim 57.

59. A method of screening for one or more compounds that are potentially useful in inhibiting or promoting muscle growth, comprising the steps of:
inserting a polynucleotide according to claim 53 into a suitable vector linked to a suitable marker gene;
transforming a suitable host cell with the vector;
administering a compound of interest to the host cell; and
determining any difference in the level of the marker gene expression.

60. The method according to claim 59, wherein the vector is selected from the group consisting of a prokaryotic plasmid, a eukaryotic plasmid and a viral vector.

61. The method according to claim 59, wherein the marker gene is a polynucleotide that encodes a protein selected from the group consisting of: a green fluorescent protein, a red fluorescent protein, a luciferase enzyme, and a β-galactosidase enzyme.

62. A method of expressing a desired protein in a muscle cell, comprising the steps of:
isolating a polynucleotide sequence that encodes the gene to be expressed;
inserting a polynucleotide according to claim 53, operably linked to the polynucleotide sequence of the protein to be expressed in a 5′-3′ orientation, into a suitable vector, and
introducing the vector into a muscle host cell.

63. The method according to claim 62, wherein the vector is selected from the group consisting of a eukaryotic vector, viral vector, and any vector suitable for gene therapy.

64. The method according to claim 62, wherein the host cell is selected from the group consisting of a primary myoblast cell line, a transformed myoblast cell line and any cell line in which the mighty promoter is active.

65. The method according to claim 62, wherein the host cell is an in vivo skeletal or cardiac muscle cell of a host animal.

66. The method according to claim 65, wherein the host animal is selected from the group consisting of a sheep, cow, deer, bull, poultry, turkey, pig, horse, mouse, rat, fish and human.
